(19) United States
(12) Patent Application Publication
(10) Pub. No.: US 2026/0240797 A1
(43) Pub. Date: Aug. 20, 2026

(54) TREATMENT OF ULCERATIVE COLITIS COMPRISING VIDOFLUDIMUS OR A PHARMACEUTICALLY ACCEPTABLE SALT THEREOF

(71) Applicant: IMMUNIC AG, Gräfelfing (DE)

(72) Inventors: Andreas MÜHLER, München (DE); Hella KOHLHOF, München (DE); Christian GEGE, Ehingen (DE); Daniel VITT, Germering (DE)

(21) Appl. No.: 18/870,046

(22) PCT Filed: May 31, 2023
(86) PCT No.: PCT/EP2023/064575

§ 371 Date: Nov. 27, 2024

(30) Foreign Application Priority Data
Jun. 1, 2022 (EP) 22176788.2
Jun. 2, 2022 (EP) 22177049.8
Apr. 4, 2023 (EP) 23166636.3

(51) Int. Cl. A61K 31/192; A61K 9/00; A61K 31/573; A61P 1/04

(52) U.S. Cl. CPC A61K 31/192; A61K 9/0053; A61K 31/573; A61P 1/04

(57) ABSTRACT

Methods of treating or ameliorating ulcerative colitis by the dihydroorotate dehydrogenase (DHODH) inhibitor vidofludimus or a pharmaceutically acceptable salt and/or a solvate, in particular a hydrate, thereof or a solvate, in particular a hydrate, of a pharmaceutically acceptable salt thereof, by administering to a human patient a therapeutically effective amount of the DHODH inhibitor. The invention also relates to methods of treating or ameliorating UC by a DHODH inhibitor without a co-medication with corticosteroids.

### SUMMARY OF THE INVENTION

The present invention relates to methods of treating or ameliorating ulcerative colitis (UC) by the dihydroorotate dehydrogenase (DHODH) inhibitor vidofludimus and/or a pharmaceutically acceptable salt and/or a solvate thereof, comprising administering to a human patient a therapeutically effective amount of the DHODH inhibitor. The invention also relates to methods of treating or ameliorating UC by a DHODH inhibitor without a co-medication with corticosteroids.

### BACKGROUND, INTRODUCTION AND PRIOR ART

Ulcerative colitis (UC) is a long-term, autoimmune disease characterized by T-cells infiltrating the colon that causes inflammation and ulceration of the colon and rectum. The main symptoms of active disease are abdominal pain and diarrhea mixed with blood. Weight loss, fever and anemia may also occur. Often, symptoms occur gradually and can range from mild to severe. Symptoms usually occur in episodes, with symptom-free periods between episodes. Complications may include abnormal enlargement of the colon (megacolon), inflammation of the eyes, joints, or liver and colon cancer.

UC can be treated with a number of medications, including 5-aminosalicylic acid (5-ASA) drugs such as sulfasalazine and mesalazine. Corticosteroids such as prednisone can also be used for their immunosuppressive and short-term curative properties, but because their risks outweigh their benefits, they are not recommended for long-term treatment. Drugs of the janus kinase (JAK) inhibitor class (e.g. tofacitinib, filgotinib) may also be used in chronic UC. Immunosuppressive drugs such as azathioprine and biologic agents (monoclonal antibodies) targeting the tumor necrosis factor (TNF), such as infliximab, adalimumab and golimumab are used only when remission cannot be achieved with 5-ASA and corticosteroids. Vedolizumab (a monoclonal antibody targeting α₄β₇ integrin) or ustekinumab (a monoclonal antibody targeting both IL-12 and IL-23) have been approved for use in adults with moderate to severe UC having a poor response to TNF blockers or corticosteroids, or for those who are steroid-dependent.

However due to their non-oral administration the compliance and persistence with biologic therapy is suboptimal.

There is still an unmet need for an effective, safe, and well tolerated treatment in subjects with UC, especially moderate to severe UC. The hallmark clinical symptoms of UC include bloody diarrhea associated with rectal urgency and tenesmus. The clinical course is marked by exacerbation and remission. The diagnosis of UC is suspected on clinical grounds and supported by diagnostic testing, and elimination of infectious causes (J. Crohn's Colitis 2012; 6:965). The most severe intestinal manifestations of UC are toxic megacolon and perforation. Extraintestinal complications include arthritis (peripheral or axial involvement), dermatological conditions (erythema nodosum, aphthous stomatitis, and pyoderma gangrenosum), inflammation of the eye (uveitis) and liver dysfunction (primary sclerosing cholangitis). Subjects with UC are at an increased risk for colon cancer and the risk increases with the duration of disease as well as extent of colon affected by the disease (Gastroenterology 2004; 126:451). The aim of medical treatment in UC is to control inflammation and reduce symptoms. Available pharmaceutical therapies are limited, do not always completely abate the inflammatory process and may have significant adverse effects. Therapies for mild to moderate active UC include 5-aminosalicylic acid derivatives and immunosuppressants. Disclosed herein is the discovery that a certain dose of vidofludimus and/or a pharmaceutically acceptable salt and/or a solvate thereof, is useful for treating UC. Accordingly, described herein are methods of reducing the severity of UC symptoms in a human subject, with more rapid onset of action and lower incidence of adverse effects.

Vidofludimus is a novel, orally available, highly selective inhibitor of dihydroorotate dehydrogenase (DHODH) and reduces the expression of proinflammatory cytokines including interleukin-17 (IL-17A and IL-17F) and interferon-gamma (IFNγ). It is in development for immune-mediated inflammatory diseases including relapsing or progressive multiple sclerosis (ClinicalTrials.gov NCT03846219, NCT05201638, NCT05134441, NCT05054140), primary sclerosing cholangitis (NCT03722576) and coronavirus disease 2019 (NCT04379271). Vidofludimus calcium has a half-life of approximately 30 h that allows for once-daily oral dosing and rapid wash-out at treatment discontinuation (Eur. J. Drug Metab. Pharmacokinet. 2020; 45:557), exhibited submicromolar potency against human DHODH and inhibited T-lymphocyte proliferation and secretion of IL-17 and IFNγ in vitro (Mult. Scler. Relat. Disord. 2020; 43:102129). In the COMPONENT trial (NCT01010581), patients with active rheumatoid arthritis treated with 35 mg vidofludimus free acid once-daily for 13 weeks had an overall similar safety profile as compared to placebo (Drugs R D 2019; 19:351). This included no notable increases in diarrhoea, alopecia, neutropenia or liver enzyme elevations.

Vidofludimus was studied in patients with inflammatory bowel disease (IBD) in an open label uncontrolled study (NCT00820365) with a confirmed diagnosis of Crohn's disease (CD) or UC. Patients were treated with a once daily 35 mg oral dose of vidofludimus over 12 weeks. Steroids were tapered during the first 8 weeks followed by a steroid-free treatment period of 4 weeks. Of the 34 patients enrolled in this trial 26 were evaluable for primary efficacy assessment. After completion of the 12 weeks treatment phase 8 out of 14 (57%) patients with CD and 6 out of 12 (50%) patients with UC were in steroid-free remission (complete responders). Another 4 (29%) patients in CD and 5 (42%) patients in UC were partial responders. Vidofludimus was well tolerated and no drug-related serious adverse events were observed (J. Crohn's Colitis 2013; 7:636).

Vidofludimus (Formula I) (2-((3-fluoro-3′-methoxy-[1,1′-biphenyl]-4-yl)carbamoyl)cyclopent-1-enecarboxylic acid) has no structural similarity to other known drugs, including leflunomide and teriflunomide. As described above, vidofludimus, in both its free acid form and its calcium salt formulation (vidofludimus calcium), has undergone clinical trials for a variety of immune-related indications. Both formulations depend on the same active substance (vidofludimus) in vivo, and thus the two formulations share the same mechanism of action, pharmacology and toxicology.

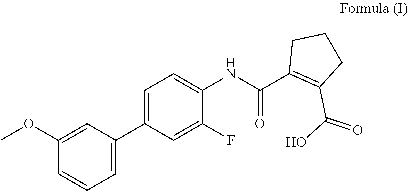 [SMILES: COc1cccc(-c2ccc(NC(=O)C3=C(C(=O)O)CCC3)c(F)c2)c1]

WO 2003/006424 and WO 2003/006425 describe certain specific compounds, which are reported to be useful for treatment and prevention of diseases where there is an advantage in inhibiting DHODH. WO 2010/128050 and WO 2015/154820 describes the use of these compounds as broad antiviral agents. WO 2012/001148 describes calcium salts of the compound of Formula (I) which inhibit DHODH and the preparation thereof (example 4). WO 2012/001151 describes other salts of the compound according to Formula (I). WO 2019/175396 describes a new white crystalline calcium salt of vidofludimus and their solvates and hydrates (designated as “Polymorph A”), a process for its preparation, a composition comprising it and its use for the treatment of chronic inflammatory and/or autoimmune diseases.

To minimize the risk of changes to uric acid during the first days of dosing with vidofludimus or a pharmaceutically acceptable salt and/or solvate thereof, a dosing regimen has been described, which use only half of the maintenance dose (WO 2019/101888).

Based on pre-clinical and clinical studies and the single and multiple-dose Phase 1 studies with vidofludimus calcium (IMU-838), daily doses of IMU-838 of up to 50 mg once daily are considered safe. The efficacy and safety profile of IMU-838 at different doses (10, 30 and 45 mg/day) was assessed in patients with relapsing remitting MS (rrMS) in the EMPhASIS study (NCT03846219). The results of this Phase 2 study demonstrated that 30 mg and 45 mg doses of IMU-838 are highly effective in patients with rrMS after 24-weeks of treatment with suppression of MRI activity by 70% for the 30 mg dose. Results also showed a favorable safety profile of IMU-838 at these doses. There was a greater decrease in serum neurofilament light chain (NfL) at 45 mg dose and this provides evidence of greater potential of neuroprotective activity for IMU-838 with increasing doses. A post-hoc analysis from EMPhASIS study in the non-relapse active subgroup of rrMS patients was conducted. The mean Expanded Disability Status Scale (EDSS) score in this subgroup of patients without any relapses during the study period has shown that a higher dose of 45 mg IMU-838 may have advantages over the 30 mg dose of IMU 838 regarding unconfirmed EDSS worsening. Based on these data and the safety profile of 45 mg IMU-838 in the EMPhASIS study, the 45 mg dose of IMU-838 is the more appropriate dose for the progressive MS population, while a daily dose of 30 mg of IMU-838 is preferred for the treatment of rrMS (U.S. Ser. No. 17/391,442, filed Aug. 2, 2021). The effect of the 10 mg dose was shown to be less efficacious than the two higher doses.

In the Phase 2 dose-finding study (NCT03341962) in patients with moderate-to-severe UC the efficacy and safety of IMU-838 for induction and maintenance therapy was assessed. The study comprises a blinded induction phase to establish the optimal dose of IMU-838 to induce response and remission, a blinded maintenance phase to evaluate the potential of IMU-838 to maintain remission until Week 50, and an open-label treatment extension arm for all patients who discontinue the blinded phase as scheduled or prematurely, subject to certain restrictions. The investigated full doses were 10, 30 and 45 mg of IMU-838 once daily for 10 to 22 weeks depending on symptomatic remission at Weeks 10 or 22. Patients received only half of their assigned full dose during the first week of treatment to minimize the risk of changes of uric acid during the first days of dosing.

We initially found a different dose response, i.e. the lowest dose of 10 mg/day IMU-838 resulted in the best efficacy outcome (e.g. higher symptomatic remission rate) compared to the higher dose of 30 and 45 mg/day. This unexpected result was in contrast to the results obtained in MS patients (see above). In addition we observed that patients without co-medication of corticosteroids benefit more of this low dose of IMU-838.

Based on new and surprisingly clinical results discussed herein in detail, without wishing to be bound by theory, it is believed that specific oral dosing, specific oral dosage forms and/or specific oral dose regimens described herein comprising the active ingredient vidofludimus and/or a pharmaceutically acceptable salt and/or a solvate, in particular a hydrate, thereof and/or a solvate, in particular a hydrate, of a pharmaceutically acceptable salt thereof, are effective in the treatment of UC as a single agent and in combination with other therapeutic agents other than corticosteroids.

In light of these findings, vidofludimus or a pharmaceutically acceptable salt or a solvate, in particular a hydrate, thereof or a solvate, in particular a hydrate, of a pharmaceutically acceptable salt thereof have the potential to be effective in treating ulcerative colitis (UC) with a potentially strong safety and tolerability profile. The results of a phase 2, multi-center, randomized, double-blind, placebo-controlled trial (NCT03341962) that investigated the safety and efficacy of vidofludimus calcium (IMU-838) as compared to placebo in patients with UC is reported which allow the determination of the therapeutically suitable effective daily dose of about 5 mg to about 15 mg, preferably about 10 mg for IMU-838 (expressed as equivalent dose relative to the anhydrous vidofludimus free acid), equally preferably a dose from about 5 mg to about 45 mg, particularly about 10 mg to about 45 mg, particularly about 30 mg to about 45 mg, particularly about 30 mg and a therapeutically suitable effective daily dose of about 12 μmol to about 38 μmol, of about 14 μmol to about 38 μmol, preferably about 28.1 μmol, equally preferably a dose of about 12 μmol to about 127 μmol, particularly a dose of about 12 μmol to about 115 μmol, particularly about 25.5 μmol to about 115 μmol, particularly about 76.5 μmol to about 115 μmol, particularly about 84.4 μmol, particularly about 76.5 μmol for vidofludimus or a pharmaceutically acceptable salt or a solvate thereof or a solvate of a pharmaceutically acceptable salt thereof.

### BRIEF SUMMARY OF THE INVENTION

The present invention is directed towards a method of treating a human patient afflicted with ulcerative colitis or presenting a clinically isolated syndrome, the method comprising orally administering to the human patient vidofludimus and/or a pharmaceutically acceptable salt thereof and/or a solvate, in particular a hydrate, thereof and/or a solvate, in particular a hydrate, of a pharmaceutically acceptable salt thereof at a daily dose of about 12 μmol to 38 μmol vidofludimus or a pharmaceutical acceptable salt thereof or solvate, in particular a hydrate, thereof or a solvate, in particular a hydrate, of a pharmaceutically acceptable salt thereof so as to thereby treat the human patient.

Especially, the efficacy of vidofludimus calcium dihydrate in the treatment of ulcerative colitis (UC), in particular in UC patients without or tapered corticosteroid co-medication, can preferably be shown by significant improvements with regard to the symptomatic remission when compared to placebo using an once-daily oral dose of vidofludimus calcium dihydrate (IMU-838) of 10 mg or 30 mg. Additionally, vidofludimus calcium dihydrate demonstrated a favorable safety and tolerability profile with this dose.

### DETAILED DESCRIPTION OF THE INVENTION

In certain embodiments the present invention relates to a method of treating ulcerative colitis as described in the following items. It will be recognized that features specified in each embodiment may be combined with other specified features to provide further embodiments of the present disclosure:

1. A method of treating a human patient afflicted with ulcerative colitis the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 12 to about 127 μmol vidofludimus or a pharmaceutically acceptable salt or a solvate, in particular a hydrate, thereof or a solvate, in particular a hydrate, of a pharmaceutically acceptable salt thereof so as to thereby treat the human patient.

2. A method of item 1 wherein the daily dose is of about 12 to about 120 μmol vidofludimus or a pharmaceutically acceptable salt or a solvate, in particular a hydrate, thereof or a solvate, in particular a hydrate, of a pharmaceutically acceptable salt thereof.

3. The method of item 1 to 2, wherein the patient is afflicted with moderate to severe ulcerative colitis.

4. The method according to any one of item 1 to 3, wherein the daily dose is about 25.5 μmol vidofludimus or a pharmaceutically acceptable salt or a solvate, in particular a hydrate, thereof or a solvate, in particular a hydrate, of a pharmaceutically acceptable salt thereof.

5. The method according to any one of item 1 to 3, wherein the daily dose is about 76.5 μmol vidofludimus or a pharmaceutically acceptable salt or a solvate, in particular a hydrate, thereof or a solvate, in particular a hydrate, of a pharmaceutically acceptable salt thereof.

6. The method according to any one of item 1 to 5, wherein the treatment is for at least 8 weeks, whereby the subject achieves clinical remission and wherein the subject achieves an endoscopic response.

7. The method of item 1 to 6, wherein the treatment is for at least 52 weeks, whereby the subject achieves clinical remission and wherein the subject achieves an endoscopic response.

8. The method according to any one of item 1 to 7, wherein clinical remission is a stool frequency subscore of less than or equal to 1, a rectal bleeding subscore of 0 and an endoscopic subscore of less than or equal to 1.

9. The method according to any one of item 1 to 8, wherein the human patient is a responder to treatment after at least 8 weeks, preferably after 52 weeks of treatment based on satisfying one or more clinical endpoints selected from the group consisting of:
(a) symptomatic remission;
(b) partial Mayo remission;
(c) Mayo rectal bleeding subscore of 0;
(d) Mayo stool frequency subscore of 0 or 1;
(e) mean absolute stool numbers decreased by at least 3;
(f) decrease in corticosteroid usage and/or dosage;
(g) corticosteroid-free symptomatic remission;
(h) corticosteroid-free partial mayo remission;
(i) normalized fecal lactoferrin;
(j) normalization of fecal calprotectin levels;
(k) >16-point improvement from induction baseline in the total Inflammatory Bowel Disease Questionnaire (IBDQ) score; and
(l) IBDQ remission.

10. The method according to any one of item 1 to 9, wherein the vidofludimus and/or a pharmaceutically acceptable salt and/or a solvate, in particular a hydrate, thereof and/or a solvate, in particular a hydrate, of a pharmaceutically acceptable salt thereof is administered as monotherapy for ulcerative colitis.

11. The method according to any one of item 1 to 9, wherein the vidofludimus and/or a pharmaceutically acceptable salt and/or a solvate, in particular a hydrate, thereof and/or a solvate, in particular a hydrate, of a pharmaceutically acceptable salt thereof is administered as adjunct therapy with another ulcerative colitis treatment.

12. The method according to any one of item 1 to 11, wherein the patient is without or tapered corticosteroid co-medication during treatment.

13. The method of item 12, wherein the patient is without corticosteroid co-medication.

14. The method according to any one of item 1 to 11, wherein the patient is no longer responding to corticosteroids.

15. The method according to any one of item 1 to 14, wherein vidofludimus is administered in the form of the vidofludimus calcium salt dihydrate with the following structure:

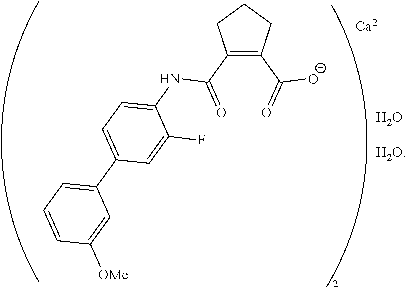

16. The method according to any one of item 1 to 15, comprising orally administering to the patient vidofludimus and/or a pharmaceutically acceptable salt thereof and/or a solvate, in particular a hydrate, thereof and/or a solvate, in particular a hydrate, of a pharmaceutically acceptable salt thereof in the form of a tablet or a capsule.

17. The method according to any one of item 1 to 16, wherein the human patient is afflicted with ulcerative colitis comprising orally administering the vidofludimus calcium salt dihydrate with the following structure:

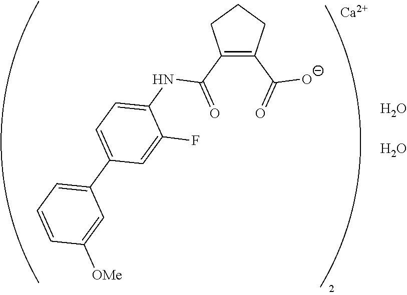

as “Polymorph A” at a daily dose of about 10 mg, or from 5 mg to 45 mg, or from 10 mg to 45 mg, or from 30 mg to 45 mg, about 30 mg or about 45 mg in the form of a tablet or a capsule.

18. The method according to any one of item 1 to 16, wherein the human patient is afflicted with ulcerative colitis comprising orally administering the vidofludimus calcium salt dihydrate with the following structure:

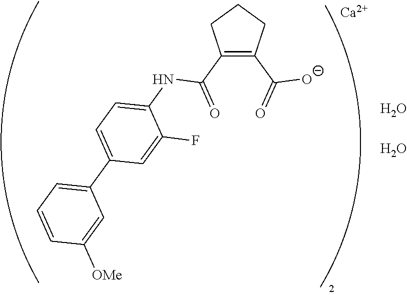

as “Polymorph A” at a daily dose of about 10 mg, or from 5 mg to 45 mg, or from 10 mg to 45 mg, or from 30 mg to 45 mg, or about 30 mg in the form of a tablet or a capsule.

19. The method according to any one of claims 1 to 16, wherein the human patient is afflicted with ulcerative colitis comprising orally administering the vidofludimus calcium salt dihydrate with the following structure:

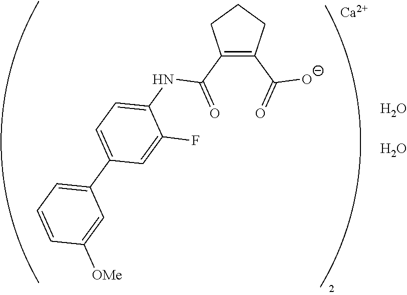

as “Polymorph A” at a daily dose of about 10 mg in the form of a tablet or a capsule.

20. The method according to any one of claims 1 to 16, wherein the human patient is afflicted with ulcerative colitis comprising orally administering the vidofludimus calcium salt dihydrate with the following structure:

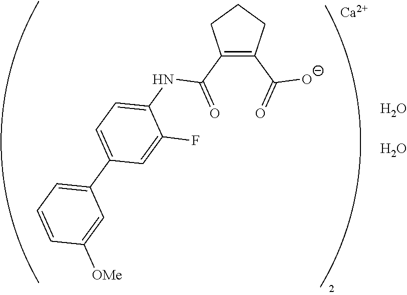

as “Polymorph A” at a daily dose of about 30 mg in the form of a tablet or a capsule.

21. The method according to any one of claims 1 to 16, wherein the human patient is afflicted with ulcerative colitis comprising orally administering the vidofludimus calcium salt dihydrate with the following structure:

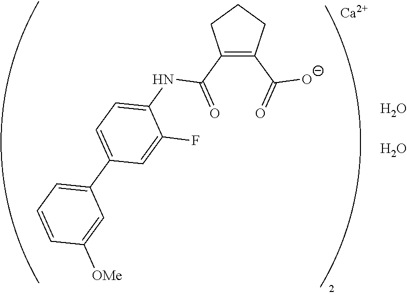

as “Polymorph A” at a daily dose of about 45 mg in the form of a tablet or a capsule.

In some embodiments, the present invention provides a method of treating a human patient afflicted with ulcerative colitis the method comprising orally administering to the human patient either
(a) vidofludimus and/or a pharmaceutically acceptable salt and/or a solvate, in particular a hydrate, thereof and/or a solvate, in particular a hydrate, of a pharmaceutically acceptable salt thereof at a daily dose of about 12 to about 127 μmol vidofludimus or a pharmaceutically acceptable salt or a solvate, in particular a hydrate, thereof or a solvate, in particular a hydrate, of a pharmaceutically acceptable salt thereof so as to thereby treat the human patient; or
(b) another DHODH inhibitor, with the proviso that the patient is without or tapered corticosteroid co-medication during treatment, preferably without corticosteroid co-medication, so as to thereby treat the human patient.

In embodiments of the present invention, corticosteroid co-medication refers in particular to corticosteroid co-medication with systemic corticosteroids, such as e.g. orally administered corticosteroids. In embodiments of the invention, corticosteroid co-medication refers in particular not to corticosteroid co-medication with non-systemic corticosteroids, such as e.g. nasally administered corticosteroids or corticosteroids administered via inhalation.

In some embodiments, another DHODH inhibitor is selected from PTC299 (emvododstat), leflunomide, teriflunomide, brequinar, BAY 2402234, MEDS433, RP7214, AG-636, ASLAN003 (farudodstat), PP-001 and JNJ-74856665, or a pharmaceutically acceptable salt and/or a solvate and/or a solvate of a pharmaceutically acceptable salt thereof.

In some embodiments, the present invention provides a method of treating a human patient afflicted with ulcerative colitis the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 12 μmol to about 120 μmol vidofludimus or a pharmaceutically acceptable salt or a solvate, in particular a hydrate, thereof or a solvate, in particular a hydrate, of a pharmaceutically acceptable salt thereof so as to thereby treat the human patient, preferably the daily dose is about 12.3 μmol to about 38.2 μmol, about 76.5 μmol to about 115 μmol, more preferably the daily dose is about 12.3 μmol to about 38.2 μmol, and most preferably the daily dose is about 26.5 μmol.

In some embodiments, the present invention provides a method of treating a human patient afflicted with ulcerative colitis the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 127 μmol vidofludimus or a pharmaceutically acceptable salt or a solvate, in particular a hydrate, thereof or a solvate, in particular a hydrate, of a pharmaceutically acceptable salt thereof so as to thereby treat the human patient.

In some embodiments, the present invention provides a method of treating a human patient afflicted with ulcerative colitis the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 115 μmol vidofludimus or a pharmaceutically acceptable salt or a solvate, in particular a hydrate, thereof or a solvate, in particular a hydrate, of a pharmaceutically acceptable salt thereof so as to thereby treat the human patient.

In some embodiments, the present invention provides a method of treating a human patient afflicted with ulcerative colitis the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 84.4 μmol vidofludimus or a pharmaceutically acceptable salt or a solvate, in particular a hydrate, thereof or a solvate, in particular a hydrate, of a pharmaceutically acceptable salt thereof so as to thereby treat the human patient.

In some embodiments, the present invention provides a method of treating a human patient afflicted with ulcerative colitis the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 76.5 μmol vidofludimus or a pharmaceutically acceptable salt or a solvate, in particular a hydrate, thereof or a solvate, in particular a hydrate, of a pharmaceutically acceptable salt thereof so as to thereby treat the human patient.

In some embodiments, the present invention provides a method of treating a human patient afflicted with ulcerative colitis the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 12 to about 38 μmol vidofludimus or a pharmaceutically acceptable salt or a solvate, in particular a hydrate, thereof or a solvate, in particular a hydrate, of a pharmaceutically acceptable salt thereof so as to thereby treat the human patient.

In some embodiments, the present invention provides a method of treating a human patient afflicted with ulcerative colitis the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 28.1 μmol vidofludimus or a pharmaceutically acceptable salt or a solvate, in particular a hydrate, thereof or a solvate, in particular a hydrate, of a pharmaceutically acceptable salt thereof so as to thereby treat the human patient.

In some embodiments, the present invention provides a method of treating a human patient afflicted with ulcerative colitis the method comprising orally administering to the human patient vidofludimus and/or a pharmaceutically acceptable salt and/or a solvate, in particular a hydrate, thereof and/or a solvate, in particular a hydrate, of a pharmaceutically acceptable salt thereof at a daily dose of about 25.5 μmol vidofludimus or a pharmaceutically acceptable salt or a solvate, in particular a hydrate, thereof or a solvate, in particular a hydrate, of a pharmaceutically acceptable salt thereof so as to thereby treat the human patient.

In some embodiments, the present invention provides a method of treating a human patient afflicted with ulcerative colitis the method comprising orally administering to the human patient vidofludimus calcium salt dihydrate with the following structure:

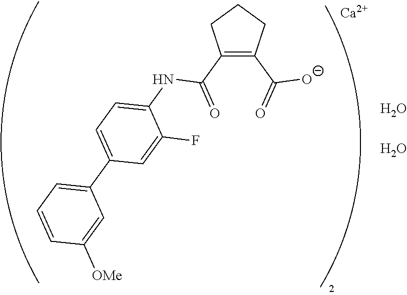

as “Polymorph A” at a daily dose of about 5 mg to about 45 mg, preferably the daily dose is about 5 mg to about 15 mg, about 30 mg to about 45 mg, more preferably the daily dose is about 5 mg to about 15 mg, and most preferably the daily dose is about 10 mg, or from 5 mg to 45 mg, or from 10 mg to 45 mg, or from 30 mg to 45 mg, or about 30 mg.

In some embodiments, the present invention provides a method of treating a human patient afflicted with ulcerative colitis the method comprising orally administering to the human patient vidofludimus calcium salt dihydrate with the following structure:

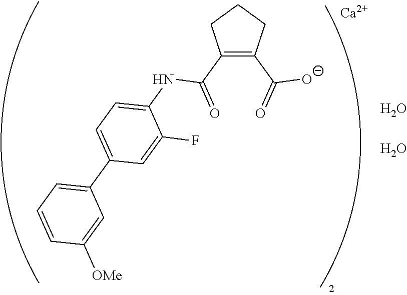

as “Polymorph A” at a daily dose of about 45 mg.

In some embodiments, the present invention provides a method of treating a human patient afflicted with ulcerative colitis the method comprising orally administering to the human patient vidofludimus calcium salt dihydrate with the following structure:

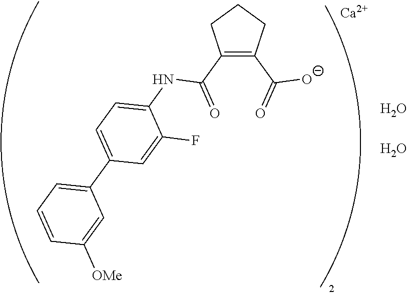

as “Polymorph A” at a daily dose of about 30 mg.

In some embodiments, the present invention provides a method of treating a human patient afflicted with ulcerative colitis the method comprising orally administering to the human patient vidofludimus calcium salt dihydrate with the following structure:

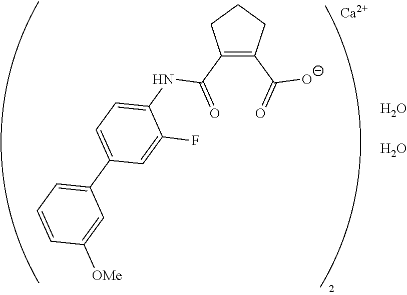

as “Polymorph A” at a daily dose of about 10 mg, or from 5 mg to 45 mg, or from 10 mg to 45 mg, or from 30 mg to 45 mg, or about 30 mg.

In some embodiments, the present invention provides a method of treating a human patient afflicted with ulcerative colitis the method comprising orally administering to the human patient vidofludimus calcium salt dihydrate with the following structure:

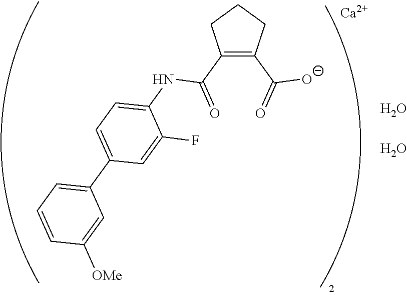

as “Polymorph A” at a daily dose of about 5 mg to about 45 mg in the form of a tablet or a capsule, preferably the daily dose is about 5 mg to about 15 mg, about 30 mg or about 45 mg, more preferably the daily dose is about 5 mg to about 15 mg, and most preferably the daily dose is about 10 mg, or from 5 mg to 45 mg, or from 10 mg to 45 mg, or from 30 mg to 45 mg, or about 30 mg.

In some embodiments, the present invention provides a method of treating a human patient afflicted with ulcerative colitis the method comprising orally administering to the human patient vidofludimus calcium salt dihydrate with the following structure:

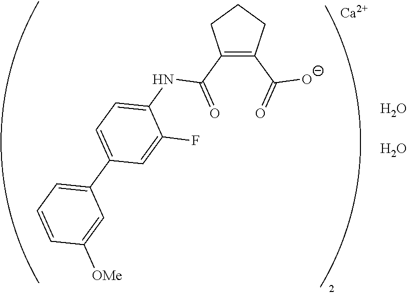

as “Polymorph A” at a daily dose of about 45 mg in the form of a tablet or a capsule.

In some embodiments, the present invention provides a method of treating a human patient afflicted with ulcerative colitis the method comprising orally administering to the human patient vidofludimus calcium salt dihydrate with the following structure:

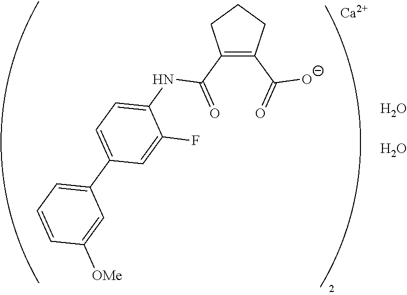

as “Polymorph A” at a daily dose of about 30 mg in the form of a tablet or a capsule.

In some embodiments, the present invention provides a method of treating a human patient afflicted with ulcerative colitis the method comprising orally administering to the human patient vidofludimus calcium salt dihydrate with the following structure:

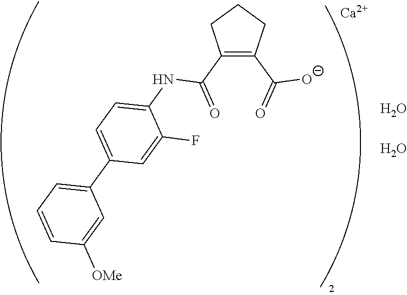

as “Polymorph A” at a daily dose of about 10 mg, or from 5 mg to 45 mg, or from 10 mg to 45 mg, or from 30 mg to 45 mg, or about 30 mg in the form of a tablet or a capsule.

Preferably, the human patient treated by methods according to embodiments of the application has had an inadequate response to or is intolerant of a conventional or existing therapy.

In some embodiments, the human patient had previously failed or was intolerant of a biologic therapy, such as an anti-TNF monoclonal antibody (e.g. infliximab, adalimumab, and golimumab) and/or vedolizumab and/or ustekinumab.

In some embodiments, the human patient had previously failed or was intolerant of a non-biologic therapy, such as a treatment with corticosteroids, JAK inhibitors (e.g tofacitinib, filgotinib), azathioprine and/or 6-mercaptopurine.

In some embodiments, the human patient had demonstrated corticosteroid dependence.

In some embodiments, the human patient is no longer responding to corticosteroids.

In certain embodiments, the human patient is a responder to a treatment of a method according to an embodiment of the application, measured preferably 52 weeks after initial treatment and after maintenance doses have been received, and is identified as having at least one of: (1) a clinical remission based on at least one of the global submissions and the US submissions; (2) an endoscopic healing; (3) a clinical response; (4) a change from baseline in Inflammatory Bowel Disease Questionnaire (IBDQ) score; (5) a mucosal healing; (6) a decrease from baseline in Mayo score; and (7) a normalization of one or more biomarkers selected from the group consisting of C-reactive protein, fecal lactoferrin and fecal calprotectin. Preferably, at least one of (1) to (7) above is identified from the subject by week 16, more preferably by week 8 or week 4, and most preferably by week 2 of the treatment.

In certain embodiments, the present invention provides an effective method of treating active UC, preferably moderately to severely active UC, in a human patient, wherein the human patient is a responder to the treatment with vidofludimus and/or a pharmaceutically acceptable salt thereof and/or a solvate, in particular a hydrate, thereof and/or a solvate, in particular a hydrate, of a pharmaceutically acceptable salt thereof, and is identified as having a statistically significant improvement in disease activity as determined by endoscopic healing with a Mayo endoscopy subscore of 0 or 1 by week 8 of treatment with vidofludimus and/or a pharmaceutically acceptable salt thereof and/or a solvate, in particular a hydrate, thereof and/or a solvate, in particular a hydrate, of a pharmaceutically acceptable salt thereof.

In other embodiments, the present invention provides an effective method of treating active UC, preferably moderately to severely active UC, in a human patient, wherein the human patient is a responder to the treatment with vidofludimus and/or a pharmaceutically acceptable salt thereof and/or a solvate, in particular a hydrate, thereof and/or a solvate, in particular a hydrate, of a pharmaceutically acceptable salt thereof, and is identified as having a statistically significant improvement in disease activity as determined by an Ulcerative Colitis Endoscopic Index of Severity (UCEIS) score of <4 by week 8 of treatment with vidofludimus and/or a pharmaceutically acceptable salt thereof and/or a solvate, in particular a hydrate, thereof and/or a solvate, in particular a hydrate, of a pharmaceutically acceptable salt thereof.

In certain embodiments, the human patient is in clinical response as determined by a decrease from baseline in the Mayo score by >30% and >3 points and a decrease from baseline in the rectal bleeding subscore>1 points or a rectal bleeding subscore of 0 or 1 by week 8 of treatment with vidofludimus and/or a pharmaceutically acceptable salt thereof and/or a solvate, in particular a hydrate, thereof and/or a solvate, in particular a hydrate, of a pharmaceutically acceptable salt thereof.

In certain embodiments, the present invention provides an effective method of treating active UC, preferably moderately to severely active UC, in a human patient, wherein a human patient identified as a non-responder to an initial treatment is administered a second treatment, preferably with an administration route different from the initial treatment. For example, a subject identified as a non-responder to an initial treatment with an IV administration of an antibody or antibody binding fragment can be treated with a subsequent oral administration of vidofludimus and/or a pharmaceutically acceptable salt thereof and/or a solvate, in particular a hydrate, thereof and/or a solvate, in particular a hydrate, of a pharmaceutically acceptable salt thereof, according to embodiments of the invention.

In certain embodiments, the present application provides a method further comprising administering to the human patient one or more additional drugs used to treat UC. In a preferred embodiment, the additional drug is selected from the group consisting of: oral 5-aminosalicylate compounds, immunomodulators, 6-mercaptopurine, azathioprine, methotrexate or a monoclonal antibody.

In certain embodiments, the present application provides a method for the administration of vidofludimus and/or a pharmaceutically acceptable salt thereof and/or a solvate, in particular a hydrate, thereof and/or a solvate, in particular a hydrate, of a pharmaceutically acceptable salt thereof as monotherapy for ulcerative colitis.

In certain embodiments, the present application provides a method for the administration of vidofludimus and/or a pharmaceutically acceptable salt thereof and/or a solvate, in particular a hydrate, thereof and/or a solvate, in particular a hydrate, of a pharmaceutically acceptable salt thereof as adjunct therapy with another ulcerative colitis treatment.

In certain embodiments, the present application provides a method for the administration to the human patient vidofludimus and/or a pharmaceutically acceptable salt thereof and/or a solvate, in particular a hydrate, thereof and/or a solvate, in particular a hydrate, of a pharmaceutically acceptable salt thereof at a daily dose of about 10 mg, or from 5 mg to 45 mg, or from 10 mg to 45 mg, or from 30 mg to 45 mg, or about 30 mg vidofludimus or a pharmaceutical acceptable salt thereof or a solvate, in particular a hydrate, thereof or a solvate, in particular a hydrate, of a pharmaceutically acceptable salt thereof so as to thereby treat the human patient.

In certain embodiments, the present application provides a method of any of the above embodiments comprising orally administering to the patient vidofludimus and/or a pharmaceutically acceptable salt thereof and/or a solvate, in particular a hydrate, thereof and/or a solvate, in particular a hydrate, of a pharmaceutically acceptable salt thereof in the form of a tablet.

In certain embodiments, the present application provides a method of any of the above embodiments comprising orally administering to the patient vidofludimus and/or a pharmaceutically acceptable salt thereof and/or a solvate, in particular a hydrate, thereof and/or a solvate, in particular a hydrate, of a pharmaceutically acceptable salt thereof in the form of a capsule.

In certain embodiments, the present application provides a method of any of the above embodiments comprising a pharmaceutical oral unit dosage form containing substantially 5 mg to 15 mg vidofludimus and/or a pharmaceutically acceptable salt thereof and/or a solvate, in particular a hydrate, thereof and/or a solvate, in particular a hydrate, of a pharmaceutically acceptable salt thereof.

In certain embodiments, the present application provides a method of any of the above embodiments comprising a pharmaceutical oral unit dosage form containing substantially 10 mg, or from 5 mg to 45 mg, or from 10 mg to 45 mg, or from 30 mg to 45 mg, or about 30 mg vidofludimus and/or a pharmaceutically acceptable salt thereof and/or a solvate, in particular a hydrate, thereof and/or a solvate, in particular a hydrate, of a pharmaceutically acceptable salt thereof.

In certain embodiments, the present application provides a method of any of the above embodiments comprising orally administering to the patient vidofludimus in the form of the vidofludimus calcium salt dihydrate with the following structure:

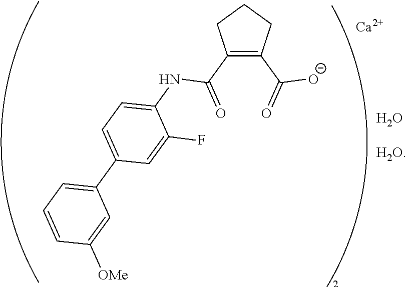

In certain embodiments the present application provides a method of any of the above embodiments comprising orally administering to the patient vidofludimus in the form of “Polymorph A” of the vidofludimus calcium salt dihydrate characterized by an X-ray powder diffraction pattern having characteristic peaks at 2 theta (±0.2°) of 5.91°, 9.64°, 16.78°, 17.81°, 19.81° and 25.41°.

In certain embodiments, the present application provides a method of any of the above embodiments comprising orally administering to the patient vidofludimus in the form of “Polymorph A” of the vidofludimus calcium salt dihydrate characterized by an FT Raman absorption spectrum having the following characteristic peaks expressed in cm⁻¹ 1664, 1624, 1617, 1532, 1449 and 1338.

In certain embodiments, the present application provides a method of any of the above embodiments comprising orally administering to the patient vidofludimus in the form of “Polymorph A” of the vidofludimus calcium salt dihydrate characterized by an IR absorption spectrum having characteristic peaks expressed in cm⁻¹ 1980, 1659, 1584, 1335 and 1145.

In certain embodiments, the present application provides a method of any of the above embodiments comprising orally administering to the patient vidofludimus in the form of a pharmaceutically acceptable salt selected from lithium, sodium, potassium, magnesium, calcium, manganese, copper, zinc, aluminium, iron, and ammonium derivatives. Salt formulations of vidofludimus and the process for preparing the same are described, e.g., in U.S. Patent Application Publication No. 2012/0035175, 2012/0029034 and 2021/0017125, and PCT International Application Publication No. WO 2012/001151, 2012/001148 and 2019/175396, which are hereby incorporated by reference into this application.

In certain embodiments, the present application provides a method wherein the dosage unit may comprise a single compound or mixtures of compounds thereof. A dosage unit can be prepared for oral dosage forms, such as tablets, capsules, pills, powders and granules.

In certain embodiments, the present application provides a method wherein vidofludimus and/or a pharmaceutically acceptable salt thereof and/or a solvate, in particular a hydrate, thereof and/or a solvate, in particular a hydrate, of a pharmaceutically acceptable salt thereof can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral administration. vidofludimus and/or a pharmaceutically acceptable salt thereof and/or a solvate, in particular a hydrate, thereof and/or a solvate, in particular a hydrate, of a pharmaceutically acceptable salt thereof can be administered alone but is generally mixed with a pharmaceutically acceptable carrier and co-administered in the form of a tablet or capsule, liposome or as an agglomerated powder. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents flow-inducing agents and melting agents.

Other aspects of the application include pharmaceutical compositions comprising vidofludimus and/or a pharmaceutically acceptable salt thereof and/or a solvate, in particular a hydrate, thereof and/or a solvate, in particular a hydrate, of a pharmaceutically acceptable salt thereof, for use in an effective method of treating active UC, preferably moderately to severely active UC, in a human patient, as well as methods of preparing the compositions and kits comprising the pharmaceutical compositions.

In certain embodiments, a kit useful for a method of the invention comprises at least one of a pharmaceutical composition for oral administration of the invention.

### Definitions

The term “vidofludimus” relates to the orally bioavailable DHODH inhibitor 2-((3-fluoro-3′-methoxy-[1,1′-biphenyl]-4-yl)carbamoyl)cyclopent-1-enecarboxylic acid) with chemical Formula (I):

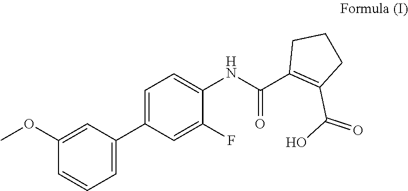 [SMILES: COc1cccc(-c2ccc(NC(=O)C3=C(C(=O)O)CCC3)c(F)c2)c1]

The preparation and characterization of pharmaceutically acceptable salts have been described in WO 2012/001151 (US 2012/0035175) and more specifically for calcium salts of vidofludimus in WO 2012/001148 (US 2012/0029034) and WO 2019/175396 (US 2021/0017125). The term “vidofludimus calcium” describes all polymorphs of the dihydrate of 1-cyclopentene-1-carboxylic acid, 2-(((3-fluoro-3′-methoxy(1,1′-biphenyl)-4-yl)amino)carbonyl)-, calcium salt (2:1) with the following chemical formula:

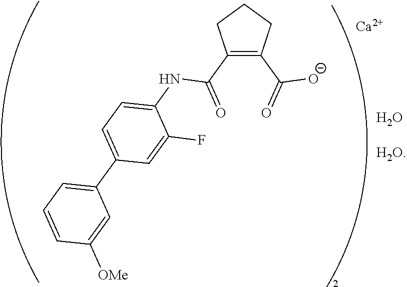

The term “IMU-838” describes one polymorph of “vidofludimus calcium” whose preparation and characterization is described in WO 2019/175396 (US 2021/0017125) as “Polymorph A”.

This “Polymorph A” is characterized by
(a) an X-ray powder diffraction pattern having characteristic peaks at 2 theta (±0.2°) of 5.91°, 9.64°, 16.78°, 17.81°, 19.81° and 25.41°; and/or
(b) an FT Raman absorption spectrum having the following characteristic peaks expressed in cm⁻¹ 1664, 1624, 1617, 1532, 1449 and 1338, and/or
(c) an IR absorption spectrum having characteristic peaks expressed in cm⁻¹ 1980, 1659, 1584, 1335 and 1145.

The term “pharmaceutically acceptable salts” refers to salts prepared from pharmaceutically acceptable non-toxic bases, including inorganic bases and organic bases. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The respective salts can be obtained by customary methods which are known to the person skilled in the art like, for example, by contacting these with an organic or inorganic base in a solvent or dispersant, or by cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts.

“Pharmaceutically acceptable” means suitable for use in a human subject.

The term “solvate” refers to a crystalline form of a molecule that further comprises molecules of a solvent or solvents incorporated into the crystalline lattice structure. Thus, the compounds of the present disclosure may be present in the form of solvates, such as those which include as solvate water, or pharmaceutically acceptable solvates, such as alcohols, in particular ethanol.

A stoichiometric or non-stoichiometric amount of solvent is bound by non-covalent intermolecular forces. When the solvent is water, the “solvate” is a “hydrate.” It is understood, that a “pharmaceutically acceptable salts” can in addition optionally contain a “solvate”.

The term “polymorph” as used herein refers to a crystalline form of a compound or a salt, or a solvate, in particular hydrate, thereof or a solvate, in particular a hydrate, of a salt thereof, in a particular crystal packing arrangement. All polymorphs have the same elemental composition.

The term “crystalline” as used herein, refers to a solid-state form which consists of orderly arrangement of structural units. Different crystalline forms of the same compound, or a salt, or a solvate, in particular a hydrate, thereof or a solvate, in particular a hydrate, of a salt thereof, arise from different packing of the molecules in the solid state, which results in different crystal symmetries and/or unit cell parameter. Different crystalline forms usually have different X-ray diffraction patterns, infrared spectra, melting points, density, hardness, crystal shape, optical and electrical properties, stability, and solubility.

“A week” preferably refers to a period of time of or about 5, about 6 or about 7 days. It may be about 5-8 days.

“A month” preferably refers to a period of time of or about 28, about 29, about 30 or about 31 days. It may be about 26-33 days.

The term “treating” or “treatment” means an alleviation of symptoms associated with a disease, disorder or condition, or halt of further progression or worsening of those symptoms.

Depending on the disease and condition of the subject, the term “treatment” as used herein may include one or more of curative, palliative and prophylactic treatment. Treatment can also include administering a pharmaceutical formulation of the present invention in combination with other therapies. Typically, a treatment according to the invention comprises an dosing-in period for about one week in which the half daily dose is administered, followed by regular treatment period in which the full daily dose of about 12 μmol to about 38 μmol, preferably about 25.5 μmol, vidofludimus and/or a pharmaceutically acceptable salt and/or a solvate, in particular a hydrate, thereof and/or a solvate, in particular a hydrate, of a pharmaceutically acceptable salt thereof is administered.

“Daily dose” preferably refers to the total dose of vidofludimus or a pharmaceutically acceptable salt or a solvate, in particular a hydrate, thereof or a solvate, in particular a hydrate, of a pharmaceutically acceptable salt thereof orally administered to the patient each day of administration. The daily dose can be reached through a single or several administrations per day, such as for example once a day, twice a day or three times a day. Preferably, it is reached or achieved by single administration per day, preferably consisting of one or more tablets or capsules, preferably tablets or capsules as described herein.

The term “moderate to severely active ulcerative colitis” is defined as having an Adapted Mayo score of 5 to 9, with an endoscopy subscore of 2 or 3.

The term “clinical remission” is based on achieving both (a) Mayo rectal bleeding subscore=0, and (b) Mayo stool frequency subscore=0 or 1, and (c) endoscopy subscore 1 or 0 on modified Mayo endoscopy score.

The term “clinical response” is a decrease from baseline in the full Mayo score of at least 3 points and at least 30%, with an accompanying decrease in the subscore for rectal bleeding of at least 1 point or an absolute subscore for rectal bleeding of 0 or 1.

The term “endoscopic remission” refers to a Mayo endoscopy subscore 0.

The term “endoscopic healing” refers to a modified Mayo endoscopy subscore of 0 or 1.

The term “endoscopic response” refers to a Mayo endoscopy subscore 0 or 1.

The term “endoscopic improvement” refers to a decrease of >1 point in Mayo endoscopy subscore or an absolute endoscopy score of <1.

The term “mucosal healing” refers to a Mayo endoscopy subscore 0 or 1 and Geboes histology score 0 or 1 (see J. Crohn's Colitis 2017; 11:305).

The abbreviation “Mayo” means the Mayo Scoring System for Assessment of Ulcerative Colitis Activity. “Adaptive Mayo Score” refers to the Adaptive Mayo Score system which has 3 subscores of the Mayo Score ranging from 0 to 9 without PGA subscore.

The term “full Mayo score” refers to the combined, partial (non-invasive) Mayo score and Mayo endoscopy score.

The term “partial Mayo score” refers to the non-invasive Mayo subscores, i.e. stool frequency, rectal bleeding and physician rating of disease activity scores.

The term “Mayo PRO-2 score” refers to the Mayo patient-reported outcome score, i.e. stool frequency and rectal bleeding score.

The term “modified Mayo endoscopy score” refers to the Mayo endoscopy score modified that a value of 1 does not include friability

The term “symptomatic response” refers to a ≥1-point decrease from Baseline in both, the Mayo rectal bleeding subscore and Mayo stool frequency subscore.

The term “symptomatic remission” refers a (a) Mayo rectal bleeding subscore=0, and (b) Mayo stool frequency subscore=0 or 1

The term “UC relapse” refers to the re-occurrence of active UC symptoms as defined as an increase of the partial Mayo score of ≥3 points for 2 consecutive weeks to a partial Mayo score of at least 5, or the need for any treatment escalation as assessed by the investigator.

Patients suffering from UC can be diagnosed in an initial diagnostic workup consisting of a complete history and physical examination, assessment of signs and symptoms, laboratory tests and endoscopy. Specific testing may include the following:
A complete blood count is done to check for anemia; thrombocytosis is occasionally seen.
Electrolyte studies and kidney function tests are done, as chronic diarrhea may be associated with hypokalemia, hypomagnesemia and kidney injury.
Liver function tests are performed to screen for bile duct involvement: primary sclerosing cholangitis.
Imaging such as X-ray or CT scan to evaluate for possible perforation or toxic megacolon.
Stool culture and Clostridioides difficile stool assay to rule out infectious colitis
Inflammatory markers, such as erythrocyte sedimentation rate or C-reactive protein.
Lower endoscopy to evaluate the rectum and distal large intestine (sigmoidoscopy) or entire colon and end of the small intestine (colonoscopy) for ulcers and inflammation.
Biopsies of the mucosa are taken during endoscopy to confirm the diagnosis of UC and differentiate it from CD. Histologic findings in UC include: distortion of crypt architecture, crypt abscesses and inflammatory cells in the mucosa (lymphocytes, plasma cells and granulocytes).

As used herein, the term “effective amount” includes a dosage sufficient to produce a desired result with respect to the indicated disorder, condition, or mental state. The desired result may comprise a subjective or objective improvement in the recipient of the dosage.

As used herein, the term “administering” includes activities associated with providing a patient an amount of vidofludimus and/or a pharmaceutically acceptable salt and/or a solvate, in particular a hydrate, thereof and/or a solvate, in particular a hydrate, of a pharmaceutically acceptable salt thereof. Administering includes providing unit dosages of compositions set forth herein to a patient in need thereof. Administering includes providing effective amounts of compounds, e.g. vidofludimus and/or a pharmaceutically acceptable salt and/or a solvate, in particular a hydrate, thereof and/or a solvate, in particular a hydrate, of a pharmaceutically acceptable salt thereof, for a specified period of time, e.g. for about 6, 9, 12, 15 or more months, or about 1, 2, 3, 4, 5 or more years.

As used herein, the term “administered as adjunct therapy” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. In some instances, the co-administered compounds are administered by the same route. In other instances, the co-administered compounds are administered via different routes. For example, one compound can be administered orally, and the other compound can be administered, e.g. sequentially or simultaneously, via intravenous or intraperitoneal injection.

The term “about” as used herein with respect to numbers, figures, ranges and/or amounts is preferably meant to mean “circa” and/or “approximately”. The meaning of those terms is well known in the art and preferably includes a variance, deviation and/or variability of the respective number, figure, range and/or amount of plus/minus 15% and especially of plus/minus 10%.

The term “another DHODH inhibitor” as used herein relates to a DHDOH inhibitor other than vidofludimus, e.g. PTC299 (emvododstat; CAS: 1256565-36-2), leflunomide (CAS: 75706-12-6), teriflunomide (CAS: 163451-81-8), brequinar (CAS: 96187-53-0), BAY 2402234 (CAS: 96187-53-0), MEDS433 (CAS: 2241027-61-0), RP7214 (CAS: 1346705-53-0), AG-636 (CAS: 1623416-31-8), ASLAN003 (farudodstat; CAS: 1035688-66-4), PP-001 (CAS: 717142-73-9), JNJ-74856665 (CAS: 2511248-11-4) and/or a pharmaceutically acceptable salt and/or a solvate and/or a solvate of a pharmaceutically acceptable salt thereof.

The term “corticosteroid co-medication” as used herein relates to treating a patient afflicted with ulcerative colitis with corticosteroids usually administered via different routes like topical (as suppositories, foams, enemas) or systemic (e.g. oral, intravenous, intramuscular) selected, without limitation, from hydrocortisone, prednisone, prednisolone, methylprednisolone, budesonide, budesonide MMX, beclomethasone dipropionate.

Compositions may further comprise one or more pharmaceutically acceptable additional ingredient(s) such as alum, stabilizers, antimicrobial agents, buffers, coloring agents, flavoring agents, adjuvants, and the like.

Compositions may be in the form of tablets or lozenges formulated in a conventional manner.

For example, tablets and capsules for oral administration may contain conventional excipients including, but not limited to, binding agents, fillers, lubricants, disintegrants and wetting agents.

Binding agents include, but are not limited to, syrup, acacia, gelatin, sorbitol, tragacanth, mucilage of starch and polyvinylpyrrolidone. Fillers include, but are not limited to, lactose, sugar, microcrystalline cellulose, maize starch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate.

Wetting agents include, but are not limited to, sodium lauryl sulfate). Tablets may be coated according to methods well known in the art.

Compositions may also be liquid formulations including, but not limited to, aqueous or oily suspensions, solutions, emulsions, syrups, and elixirs. The compositions may also be formulated as a dry product for constitution with water or other suitable vehicle before use, such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminium stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

### EXAMPLES

### Example 1: A Phase 2, Multicenter, Randomized, Double-blind, Placebo-controlled, Dose-finding Study to Evaluate the Efficacy and Safety of IMU-838 for Induction and Maintenance Therapy in Moderate-to-severe Ulcerative Colitis (CALDOSE-1)

(CALDOSE-1) is a clinical trial registered with ClinicalTrials.gov (NCT03341962).

### Objectives: Primary Objective

To determine the optimal dose of IMU-838 for the induction of symptomatic remission (based on Mayo PRO-2 score) and endoscopic healing (based on Modified Mayo endoscopy score) in patients with moderate-to-severe ulcerative colitis (UC) Secondary objectives
To evaluate the efficacy of IMU-838 in patients with moderate-to-severe UC
To evaluate the potential of IMU-838 to induce response and remission by Week 10
   To evaluate the potential of IMU-838 to maintain remission through Week 52
   To evaluate the safety and tolerability of IMU-838 in patients with moderate-to-severe UC

### Definitions

Full Mayo score: Combined, partial (non-invasive) Mayo score and Mayo endoscopy score
Partial Mayo score: Non-invasive Mayo subscores, i.e. stool frequency, rectal bleeding, and physician rating of disease activity scores
Mayo PRO-2 score: Mayo patient-reported outcome score, i.e. stool frequency and rectal bleeding score
Modified Mayo endoscopy score: The Mayo endoscopy score modified that a value of 1 does not include friability Symptomatic response: ≥1-point decrease from Baseline in both, the Mayo rectal bleeding subscore and Mayo stool frequency subscore
Clinical response: Decrease from Baseline in the full Mayo score of at least 3 points and at least 30%, with an accompanying decrease in the subscore for rectal bleeding of at least 1 point or an absolute subscore for rectal bleeding of 0 or 1
Symptomatic remission: ⋅ Mayo rectal bleeding subscore=0, and
   Mayo stool frequency subscore=0 or 1
Clinical remission: ⋅ Mayo rectal bleeding subscore=0, and
   Mayo stool frequency subscore=0 or 1, and
   Endoscopy subscore 1 or 0 on modified Mayo endoscopy score
Endoscopic Modified healing: Mayo endoscopy subscore of 0 or 1
UC relapse: Re-occurrence of active UC symptoms as defined as an increase of the Partial Mayo score of ≥3 points for 2 consecutive weeks to a Partial Mayo score of at least 5, or the need for any treatment escalation as assessed by the investigator
Design: Overview: This is a phase 2, multicenter, randomized, double-blinded, and placebo-controlled trial in patients with moderate-to-severe UC with an option for open-label treatment extension. The study comprises a blinded induction phase to establish the optimal dose of IMU-838 to induce response and remission, a blinded maintenance phase to evaluate the potential of IMU-838 to maintain remission until Week 52, and an open-label treatment extension arm for all patients who discontinue the blinded phase as scheduled or prematurely.
   Blinding to individual treatment assignments during the induction and maintenance phase will be maintained throughout both phases for patients, investigators, and other personnel involved in the conduct of this trial. Patients that either discontinued treatment in the blinded treatment arms or that have completed an overall 52 weeks of blinded treatment have the option to enter an open-label extension arm of this study.
   The primary efficacy endpoint will be assessed at Week 10 of the induction phase.
Induction phase: Patients with a confirmed diagnosis of moderate-to-severely active UC will be included and randomized to treatment. Diagnosis of UC must have been established at least 3 months before enrollment by clinical and endoscopic evidence, and endoscopic confirmation of disease activity (colonoscopy within 1 year of enrollment and flexible sigmoidoscopy at Screening). Randomization will be stratified by prior use of any biologics and current use of corticosteroids. In addition the proportion of patients who had received previous biologics will be limited to 75% of all included patients. The induction phase will have a 2-stage design:
   In Enrollment Period 1 approximately 60 patients will be randomized 1:1:1:1 to oral 10 mg/day, 30 mg/day, or 45 mg/day IMU-838, or placebo (15 patients each).
   In Enrollment Period 2, approximately 135 patients will be randomized 1:1:1 to two doses of IMU-838 or placebo (about 45 patients in each group). The IMU-838 doses used will correspond to those two consecutive IMU-838 doses used in Enrollment Period 1 that show better activity than a lower IMU-838 dose or placebo in terms of efficacy and PD with acceptable tolerability as evaluated in the interim analysis.
   Recruitment into Enrollment Period 1 will continue until results of the interim analysis will be available and Enrollment Period 2 will be started.
   A flexible sigmoidoscopy with biopsy will be performed at Screening. If a full colonoscopy was not performed in the previous year, the screening sigmoidoscopy should be replaced by a full colonoscopy.
   The induction phase (both enrollment periods) will be at least 10 weeks. During the induction phase safety, PD, the partial Mayo score (and respective subscores), IMU-838 blood levels, and assessment of biomarkers will be regularly evaluated.
   At Week 10, the assessment for the primary efficacy endpoint will be made, including a flexible sigmoidoscopy with biopsy and assessment of clinical scores. If any patient is unable to reach Week 10 due to disease activity, adverse events or other reasons, the patient has the option to continue in the open-label treatment extension arm, if the patients had at least 2 valid assessment of the Mayo PRO-2 score post-baseline, and had received at least 6 weeks of treatment without any major protocol deviations of the dosing schedule. When the patient decides to enter the open-label treatment extension arm, the Week 10 sigmoidoscopy should be performed at that time of switch or arm, if possible.
   Results of all sigmoidoscopy assessments (and screening colonoscopy assessment if applicable) will be evaluated by an independent central reader, who will also assess the modified Mayo endoscopy score and will confirm endoscopic eligibility, before a patient will be randomized.
   After Week 10, patients will be treated as follows:
      Patients who have achieved symptomatic remission at Week 10 will enter the maintenance phase.
      Patients who have achieved symptomatic response but not yet symptomatic remission, will continue into an extended induction phase up to Week 22 (with assessments of response and remission every 4 weeks). Patients can enter the maintenance phase at any time if they have reached symptomatic remission.
      Patients who do not show symptomatic response at Week 10 or any time of the extended induction phase if applicable will be discontinued from the blinded treatment but will have the option to enroll in the open-label treatment extension arm.
   Any concomitant medication allowed in the induction phase should be maintained at a constant dose throughout the induction phase, including a potential extended induction period.
Maintenance phase: All patients showing symptomatic remission during the induction phase at Week 10, or during the extended induction phase between Weeks 10 and 22, will receive maintenance treatment as follows:
   patients who had received IMU-838 during induction will be re-randomized to 10 mg/day or 30 mg/day IMU-838
   patients who had received placebo will continue placebo treatment
   the respective treatment will be administered until the patient has received IMU-838 or placebo treatment for a total of 52 weeks (adding up induction phase, potential extended induction phase and maintenance treatment) or until UC relapse, whatever occurs first.
   Randomization will be stratified by IMU-838 dose received in the induction phase, prior use of any biologics, and current use of corticosteroids.
   Safety (safety laboratory and assessment of adverse events) and partial Mayo score (including respective subscores) will be regularly assessed during the maintenance phase. Patient visits in the maintenance phase will be scheduled monthly. All patients who remain in the maintenance phase until Week 52 will undergo a scheduled flexible sigmoidoscopy with biopsy and will have the full Mayo score (including all subscores) assessed.
   If a patient discontinues from the blinded maintenance phase (due to UC relapse, adverse event or other reasons), a flexible sigmoidoscopy with biopsy will be performed at the time of treatment discontinuation, unless the discontinuation occurs within 4 weeks of the last endoscopy examination during this trial. The patient has the option to enter the open-label treatment extension arm, if fulfilling inclusion criteria for this arm.
   An interim analysis of the maintenance phase will be performed at the time of the final analysis for the induction phase (i.e. when all patients in both enrollment periods of the induction phase have completed Week 10) for the following reasons:
      to support planning of further IMU-838 development, including Phase 3 studies, and
      to evaluate if the remaining maintenance phase must be adjusted to accommodate for observed relapse rates.
   The final analysis for the maintenance phase will be performed when all patients enrolled in the maintenance phase have terminated maintenance treatment i.e. have completed 52 weeks of treatment or had an UC relapse, whatever comes first.
Open-label treatment extension arm: To enter the open-label treatment extension arm directly from the induction phase, patients must have at least 2 valid Mayo PRO-2 score assessments post-baseline, and must have received treatment for at least 6 weeks without any major protocol deviations of the dosing schedule. Patients can enter the open-label treatment extension arm from the maintenance phase at any time, or the conclusion of a total 52 weeks of treatment. The blinding of the randomized treatment will always be maintained for patients entering the open-label extension arm.
   Patients who have ongoing significant treatment-emergent adverse events, had significant protocol deviations or had shown significant non-compliance will be excluded from the open-label extension arm. If ongoing treatment-emergent adverse events were the reason for exclusion from the open-label extension arm, the eligibility can be re-assessed up to 4 weeks following the last treatment in the blinded treatment arms.
   All patients will receive a daily dose of 30 mg IMU-838 in the open-label treatment extension arms. The Sponsor of this trial reserves the right to change the dose whenever additional information regarding safety and efficacy of IMU-838 becomes available.
   Patient visits will be scheduled monthly for the initial 52 weeks of study treatment, and every 3 months thereafter.
   Adverse events, concomitant medication, vital signs, partial Mayo score, IMU-838 blood concentrations, fecal calprotectin, and C-reactive protein, as well as a limited number of safety laboratory and urinalysis parameters will be regularly assessed during clinic visits.
   No endoscopic assessment will be performed but results from such procedures when done in the course of routine clinical care will be collected.
   Patients can continue treatment as long as there are no toxicities that would be a clinically significant safety issue for this patient as assessed by the investigator, or until the patient withdraws consent.
Study termination: The study will be terminated when all patients have discontinued treatment in the induction phase, maintenance phase, and in the open-label extension arm, or when the Sponsor decides to close the study for any reason, whatever comes first.

### Treatments:

Medicinal product under investigation: IMU-838 (vidofludimus calcium)
   Formulation: Tablets with 5 mg, 15 mg, and 22.5 mg IMU-838
   Doses: induction phase: 10 mg/day (2×5 mg tablets)
      30 mg/day (2×15 mg tablets)
      45 mg/day (2×22.5 mg tablets)
   maintenance phase: 10 mg/day (2×5 mg tablets)
      30 mg/day (2×15 mg tablets)
   open-label arm: 30 mg/day (2×15 mg tablets)
   Frequency: Once daily
Reference: Placebo, 2 tablets once daily
Treatment duration: Induction phase: At least 10 weeks and up to 22 weeks, depending on when symptomatic remission is achieved.
   Maintenance phase: Until 52 weeks of total study treatment (adding up induction phase, potential extended induction phase, and maintenance treatment) or until relapse (re-occurrence of active UC symptoms), whatever occurs earlier.
   Open-label extension arm: As long as there are no toxicities that would be a clinically significant safety issue for the patient as assessed by the investigator, or until the patient withdraws consent, or until study termination by the Sponsor.
Diagnosis and main inclusion criteria: Induction phase
   1. Male and female patients, aged 18-80 years
   2. Moderate-to-severe UC, diagnosed more than 3 months before Screening
   3. Previous treatment failure defined as:
   Patient had an inadequate response with, lost response to, or was intolerant to biologics (no more than 2 treatment failures with biologic drugs) or immunomodulators; or
   Patient had an inadequate response with, was intolerant to, or demonstrated dependence on corticosteroids
   4. Active disease as defined by a full Mayo score≥6, with modified mayo endoscopy subscore of 2 or 3 at screening flexible sigmoidoscopy (endoscopy assessed by an independent central reader blinded to screening center and patient information)
   5. UC extending >15 cm from the anal verge as confirmed by an independent central reader (blinded to screening center and patient information)
   6. Laboratory values: Neutrophil count>1500 cells/μL, platelet count≥100 000/mm³, serum creatinine<1.5 upper limit of normal (ULN), total bilirubin, alanine aminotransferase, and aspartate aminotransferase<1.5 ULN
   7. Women of childbearing potential must have a negative pregnancy test at Screening (blood test) and Visit 1 (if not the same day as Screening, urine test)). They must agree not to attempt to become pregnant, must not donate ova, and must use a highly effective contraceptive method 1 month before start of the study, during treatment with IMU-838, and at least 60 days after the last dose of study therapy
   8. Male patients must agree not to father a child or to donate sperm and use adequate barrier contraception during treatment with IMU-838 and for at least 60 days after the last dose of study therapy
   9. Ability to understand and comply with study procedures and restrictions
   10. Written informed consent
   Maintenance phase
   1. Symptomatic remission achieved during the induction phase
   Open-label treatment extension arm
   1. Patient had received at least 6 weeks of treatment without any major protocol deviations of the dosing schedule
   2. Patient had at least 2 valid assessments of the Mayo PRO-2 score post-baseline
Main exclusion criteria: 1. Ulcerative proctitis (ulceration restricted to the distal 15 cm of the large intestine)
   2. Diagnosis of Crohn's Disease, inflammatory bowel disease type unclassified, ischemic colitis, microscopic colitis, radiation colitis or diverticular disease-associated colitis
   3. Ileostomy, colostomy, or known fixed symptomatic stenosis of the intestine
   4. History of colectomy with ileorectal anastomosis or ileal-pouch anal anastomosis
   5. Active therapeutically uncontrollable abscess or toxic megacolon
   6. Malabsorption or short bowel syndrome
   7. Evidence of, or treatment for Clostridium difficile infection or other intestinal pathogen within 30 days prior to enrollment
   8. Currently active, clinically significant systemic infection(s) (e.g. tuberculosis, HIV, hepatitis B or C)
   9. History of colorectal cancer or colorectal dysplasia (with the exception of dysplasia in polyps which have been removed)
   10. History of, or current serious, severe, or unstable (acute or progressive) physical or mental illness, or any medical condition, including laboratory anomalies or renal or hepatic impairment, that may require treatment or would put the patient on jeopardy if he/she was to participate in the study or would confound interpretation of study data
   11. History of renal diseases, especially those favoring or resulting in nephrolithiasis (including known history of nephrolithiasis, hyperoxalemia, or hyperuricemia)
   12. Serum uric acid levels at Screening>1.2 ULN
   13. Known hypothyroidism and/or diabetes
   14. History of gout or symptoms suggestive of gout
   15. History of multiple urinary tract infections
   16. Known or suspected Gilbert's syndrome (Morbus Meulengracht)
   17. Known hypersensitivity to DHODH inhibitors or any ingredient of the investigational product
   18. Pregnancy or breastfeeding
   19. History of drug or alcohol abuse during the past year
   20. Use of prohibited medication
   21. Use of any other investigational agent or participation in any other clinical trial within the last 8 weeks or 5×the half life of the respective investigational agent before Screening, whatever is longer
   Additional exclusion criteria for open-label arm
   1. Any ongoing, clinically significant (as assessed by the investigator) adverse event or laboratory abnormality (including blood chemistry and urinalysis) that is treatment-emergent (started during the IMU-838 treatment in the blinded treatment arms)*
   2. Significant treatment or study non-compliance during induction and/or maintenance phase (as assessed by the investigator), and/or inability or unwillingness to follow instructions by study personnel
   3. Significant protocol deviations during induction and/or maintenance phase that are assessed by the investigator to negatively affect patient compliance in this study
      If continuing treatment-emergent adverse events are the reason for exclusion from the open-label extension arm, the eligibility can be re-assessed up to 4 weeks following the last treatment in the blinded treatment arms.
Concomitant medication: The following medications will be prohibited during the entire clinical trial (during the induction and maintenance phase and open-label treatment extension) and, if applicable, before Screening as indicated:
   Any investigational product within 8 weeks or 5×the respective half life before Screening, whatever is longer, and during the entire trial
   Thiopurines (e.g. azathioprine and 6-mercaptopurine) to be discontinued at least 2 weeks before Screening
   Intravenous corticosteroids to be discontinued at least 4 weeks before Screening
   Use of any DHODH inhibitor (e.g. leflunomide or teriflunomide) within 6 months of Screening
   Any use of natalizumab (Tysabri™) within the 12 months before Screening
   Use of cyclosporine, methotrexate, mycophenolate mofetil, or any calcineurin inhibitors (e.g. tacrolimus or pimecrolimus)
   Medications that are indicated to influence diuresis
   Any active treatments for malignant disease
   The following medication are allowed but with the following conditions:
      Oral corticosteroids must have been discontinued at least 2 weeks before Screening with the following exceptions
         Oral systemic corticosteroids (at or below 20 mg/day prednisolone equivalent), including budesonide (MMX formulation, at or below 9 mg/day) and beclomethasone dipropionate, will be allowed if they had been used for at least 4 weeks and with a stable dose for at least 2 weeks before Screening, however:
         Cannot be initiated during induction or maintenance phase, allowed only if already taken at screening at stable doses≤20 mg/day
         Doses should be stable throughout induction phase
         Dose can be changed during maintenance phase but must be maintained ≤20 mg/day, weaning should be attempted
         Oral corticosteroid treatment can be continued or newly initiated during open-label extension, however must be maintained ≤20 mg/day
      Oral aminosalicylates (e.g. mesalazines) must have been discontinued at least 2 weeks prior to Screening with the following exception:
         Oral aminosalicylates will be allowed if they had been used for at least 6 weeks and with a stable dose for at least 3 weeks before Screening
         Cannot be initiated during induction or maintenance phase, allowed only if already taken at screening at stable doses
         Doses should be stable throughout induction phase
         Dose can be changed during maintenance phase but must be maintained ≤4 g/day
         Oral aminosalicylates treatment can be continued or newly initiated during open-label extension, however must be maintained ≤4 g/day
      Biologics must have been discontinued before Screening as follows: anti-tumor necrosis factor a antibodies (e.g. infliximab, adalimumab, golimumab) within 8 weeks; vedolizumab and ustekinumab within 16 weeks (but can be newly initiated during open-label extension phase, if needed)
   The following medications are prohibited at Screening and during the induction phase, but are allowed during the maintenance phase and during open-label extension:
      Topical treatments (enemas or suppositories) containing aminosalicylates and/or budesonide
      Oral and/or systemic antibiotic treatments unless used for a Clostridium difficile infection or other bacterial infections can be used in maintenance phase and throughout open-label extension
   The dosage of any allowed concomitant medication must be stable throughout the induction phase (including during any extended induction period), however they can be modified thereafter.
Endpoints:
Primary: Induction phase
   Composite endpoint: Proportion of patients with both, symptomatic remission (based on Mayo PRO-2 score) and endoscopic healing (based on Modified Mayo endoscopy score) at Week 10
      All endoscopic subscores will be assessed by an independent central reader blinded to treatment
      All patients (both, Enrollment Period 1 and Enrollment Period 2 patients) who have received the active doses selected for Enrollment Period 2 will be combined (N=120) and compared with placebo (N=60)
Secondary: Induction phase
Efficacy:
   Proportion of patients with both, symptomatic remission and endoscopic healing at Week 10 for the following comparisons:
      the higher active dose versus placebo
      the lower active dose versus placebo
      the lower versus the higher active dose
   Proportion of patients achieving symptomatic remission during the induction phase
   Proportion of patients with clinical response at Week 10
   Proportion of patients with endoscopic healing at Week 10
   Proportion of patients with symptomatic response during the induction phase
   Time course of Mayo scores (full score, partial score, Mayo PRO-2 score) over 10 weeks and changes from Baseline
   Time course of fecal calprotectin and C-reactive protein
   Changes from Baseline in PD parameters (ex-vivo lymphocyte stimulation, whole blood gene expression, blood cytokines, miR-122 expression, blood genotyping, bacterial peptides, tissue based markers) at Week 10
   Maintenance phase
      Proportion of patients in symptomatic remission by visit and at Week 52
      Time to UC relapse
      Proportion of patients without relapse following 22 and 52 weeks of continuous maintenance therapy
      Time course of fecal calprotectin and C-reactive protein
      Endoscopic and microscopic healing at Week 52
      Corticosteroid-free remission at Week 52 in patients receiving corticosteroids at Baseline
   Open-label treatment extension arm
      Proportion of patients with symptom control
      Time course of fecal calprotectin and C-reactive protein
Safety: ⋅ Incidence and severity of adverse events
   Physical examination, body weight, and vital signs (only induction and maintenance phase)
   Electrocardiogram (12-lead ECG)*
   Safety laboratory: hematology, clinical chemistry, coagulation parameters, urinalysis, liver function tests
Pharmaco-kinetics: Induction and maintenance phase, open label extension arm
   Blood levels of IMU-838

### Induction Phase Results

The demographics, i.e. the stratification at randomization for prior biologics and concurrent corticosteroid use the induction phase (see FIG. 1) data of this Phase 2 trial is shown in FIG. 2. Clinical remission values (FIG. 3) indicate that the primary endpoint was not achieved, however that the clinical remission at Week 10 was best within the lowest dose of IMU-838 (10 mg/day) compared to higher doses (30 and 45 mg/day). As shown in FIG. 4 and FIG. 5, without concurrent use of corticosteroids the clinical remission can be improved. FIG. 6 indicates, that the lowest dose of IMU-838 (10 mg/day) compared to higher doses (30 and 45 mg/day) is beneficial in endoscopic healing, clinical remission and symptomatic response.

The trial did not achieve the primary endpoint of clinical remission for the pooled 30 and 45 mg/day active dose groups of vidofludimus calcium versus placebo at week 10. In addition, no meaningful differences were observed between the three active dose groups for the overall intent-to-treat patient population (10 mg/day: 14.9%, 30 mg/day: 10.6%, 45 mg/day: 13.6%, placebo: 12.5%) or for the trial's other secondary endpoints, including symptomatic remission, or endoscopic healing.

Consistent with prior data sets in other patient populations, administration of vidofludimus calcium in this trial was observed to be safe and well-tolerated. No new safety signals were observed. As compared to placebo, there were no increased rates of infections and infestations, no elevated rates of liver events or liver enzyme elevations, and no elevated rates for changes in hematology-related laboratory variables. The most common adverse events in this trial were anemia (15/263 patients, 5.7%), headache (9/263 patients, 3.4%) and COVID-19 (7/263 patients, 2.7%). Most adverse events were generally mild in severity. As is common for the design of clinical trials in UC, the use of oral systemic corticosteroids (≤20 mg/day prednisolone equivalent) was allowed in CALDOSE-1 in patients who had been treated with corticosteroids for at least four weeks before randomization. Doses of corticosteroids were required to be kept constant throughout the induction phase (weaning was not allowed in this phase of the trial), and the distribution of patients using corticosteroids was equal throughout all treatment groups.

Surprisingly, CALDOSE-1 data suggest a previously unknown treatment interference between the efficacy of vidofludimus calcium and the concurrent use of corticosteroids in the UC patient population. More specifically, the non-steroid patient population showed an 11.4% advantage in clinical remission for vidofludimus calcium over placebo (pooled vidofludimus calcium treatment groups at week 10: 14.7%, placebo: 3.3%). Such a difference in clinical remission between active treatment and placebo would traditionally be considered as confirming therapeutic activity. In contrast, patients concomitantly taking vidofludimus calcium and corticosteroids during induction treatment had a lower rate of clinical remission at week 10 (11.5%) than placebo patients (20.6%) and also lower than the group of vidofludimus calcium monotherapy without concurrent use of steroids (14.7%). This treatment interference between vidofludimus calcium and corticosteroids was not expected by currently available preclinical or clinical data. Multivariate analysis shows that concomitant steroids have highest impact on clinical remission data:

| Parameter | Estimate | P-value |
|---|---|---|
| Concomitant use of | 0.59 | 0.057 |
| corticosteroids | | |
| Full Mayo score at baseline | −0.29 | 0.097 |
| Time from diagnosis | 0.02 | 0.505 |
| Prior use of biologics | −0.12 | 0.732 |

Multivariate logistical regression analysis for endpoint of Clinical Remission; use of four dependent variables, interaction term and pooled treatment groups of 30/45 mg, full analysis set (N₁₀ = 67, N₃₀ = 66, N₄₅ = 66, N_{placebo} = 64

This yields an estimation of odd ratios for vidofludimus calcium achieving clinical remission versus placebo:

| | Odds Ratio Estimate |
|---|---|
| Active treatment groups of 30/45 mg to | 3.53 |
| achieve clinical remission versus placebo | |
| without concomitant steroids | |
| Active treatment groups of 30/45 mg to | 0.58 |
| achieve clinical remission versus placebo | |
| with concomitant steroids | |

### Example 2: Content of Different Doses of Vidofludimus

As described above, vidofludimus, in both its free acid form and its calcium salt formulation (vidofludimus calcium), has undergone clinical trials for a variety of immune-related indications. Both formulations depend on the same active substance (vidofludimus) in vivo, and thus the two formulations share the same mechanism of action, pharmacology and toxicology. IMU-838 is the “Polymorph A” of the dihydrate of 1-cyclopentene-1-carboxylic acid, 2-(((3-fluoro-3′-methoxy(1,1′-biphenyl)-4-yl)amino)carbonyl)-, calcium salt (2:1), characterized by an X-ray powder diffraction pattern having characteristic peaks at 2 theta (±0.2°) of 5.91°, 9.64°, 16.78°, 17.81°, 19.81° and 25.41°. The preparation of this “Polymorph A” is described in WO 2019/175396, which is incorporated herein by this reference.

In the following Table 1 the amount (in mg) of active moiety of the compound is converted into μmol.

TABLE 1

| | | mg vidofludimus |
|---|---|---|
| mg IMU-838 | μmol IMU-838 | (free acid) |
| 55.2 | 141 | 50 |
| 49.7 | 127 | 45 |
| 45 | 115 | 41 |
| 33.1 | 84.4 | 30 |
| 30 | 76.5 | 27 |
| 24.9 | 63.3 | 22.5 |
| 22.1 | 56.3 | 20 |
| 16.6 | 42.2 | 15 |
| 15 | 38.2 | 14 |
| 11.0 | 28.1 | 10 |
| 10 | 25.5 | 9.1 |
| 5.5 | 14.1 | 5.0 |
| 5 | 12.3 | 4.5 |

The numerical values given in the description, examples and claims are expressed as equivalent dose relative to the anhydrous vidofludimus free acid (i.e., the active moiety).

### Example 3: Outcome of the CALDOSE-1 Clinical Study (Week 50)

Week 50 patient data (final analysis maintenance phase (MP)) from the CALDOSE-1 clinical study as described above was analysed from the following patients (per treatment group). FIG. 7 shows the patient population in the maintenance phase (MP) for each group.

The results were as follows:

Clinical remission was determined as composite of patient-reported symptomatic remission (stool frequency Mayo subscore of 0 or 1, rectal bleeding Mayo score of 0) and modified Mayo endoscopy subscore of 0 or 1. The results are shown in Table 2.

TABLE 2
Clinical Remission at Week 50

| | | 10 mg | 30 mg |
|---|---|---|---|
| | Placebo | IMU-838 | IMU-838 |
| Data Set: (n-Evaluated/n-Total of group) | (n = 18/21) | (n = 26/35) | (n = 26/29) |
| Number of Patients with Clinical | 5 | 11 | 16 |
| Remission at Week 50 | | | |
| Clinical Remission Rate at Week 50 | 27.8% | 42.3% | 61.5% |

Full Analysis Set MP: all patients randomized into maintenance phase (N_{placebo} = 27, N₁₀ = 45, N₃₀ = 40)

Both 10 and 30 mg IMU-838 showed improvement of the patients over placebo, and a statistically significant difference was found in favor of 30 mg IMU-838 vs. Placebo for achieving clinical remission at Week 50, with a (Two-sided Pearson's chi-square test (significance level alpha=0.05)), with a P-value (2-sided) of p=0.0358, an odds ratio (30 mg IMU-838/Placebo) of 4.16, and a delta (Δ) of 33.8% with a confidence interval (95% CI) of 3.2%-59.3%.

The results for steroid-free clinical and symptomatic remission at Week 50 (no receipt of systemic or local corticosteroids during the study) receiving corticosteroids at induction-baseline are shown in Table 3.

TABLE 3
Clinical Remission at Week 50 in steroid-free patients

| | | 10 mg | 30 mg |
|---|---|---|---|
| Data Set | Placebo | IMU-838 | IMU-838 |
| Number of Patients with Clinical | 3 | 5 | 8 |
| Remission at Week 50 | | | |
| Clinical Remission Rate at Week 50 | 27.3% | 38.5% | 66.7% |

Modified Full Analysis Set MP: all patients that were randomized into maintenance phase who received concomitant corticosteroids during induction phase (N₁₀ = 22, N₃₀ = 22, N_{placebo} = 17)

Both 10 and 30 mg IMU-838 showed improvement of over placebo in steroid-free patients, with a markedly higher improvement in the 30 mg group.

The results for endoscopic healing at Week 50 (endoscopic improvement acc. FDA definition: modified Mayo endoscopy subscore of 0 or 1) are shown in Table 4.

TABLE 4
Endoscopic Healing at Week 50

| | | 10 mg | 30 mg |
|---|---|---|---|
| | Placebo | IMU-838 | IMU-838 |
| Data Set: (n-Evaluated/n-Total of group) | (n = 17/21) | (n = 28/35) | (n = 26/29) |
| Number of Patients Meeting Endpoint | 6 | 15 | 19 |
| Endoscopic Healing Rate at Week 50 | 35.3% | 53.6% | 73.1% |

Full Analysis Set MP: all patients randomized into maintenance phase (N₁₀ = 45, N₃₀ = 40, N_{placebo} = 27)

Both 10 and 30 mg IMU-838 showed improvement of over placebo in the endoscopic healing with a markedly higher improvement in the 30 mg group.

Results for symptomatic UC relapse (defined as re-occurrence of active UC symptoms defined as an ≥2-point increase from the Week 10 value (or Week 22 value if the patient received remission only during the extended induction phase) in Mayo PRO-2 score for 2 consecutive visits, or the need for any treatment escalation as assessed by the investigator after excluding intermittent infections (at the investigator's conclusion)) are shown in Table 5.

TABLE 5
Time to symptomatic UC relapse

| | | 10 mg | 30 mg |
|---|---|---|---|
| Data Set: (n-Evaluated/n-Total of | Placebo | IMU-838 | IMU-838 |
| group) | (N = 27) | (N = 45) | (N = 40) |
| Mean time to symptomatic UC | 186.6 | 231.7 | 221.9 |
| relapse [days]° | | | |

Both 10 and 30 mg IMU-838 delayed the next symptomatic relapse as compared to placebo.

In summary, the maintenance phase results of the CALDOSE-1 clinical study demonstrate that IMU-838 provides a benefit regarding Clinical Remission (as compared to placebo) in UC patients without concurrent use of corticosteroids. At Week 50, the dose of 30 mg IMU-838 once-daily demonstrated a statistically significant higher proportion of patients (p=0.0358) achieving Clinical Remission versus placebo treatment. IMU-838 delayed symptomatic UC relapse as compared to placebo.

For most efficacy endpoints, a dose-linear response was observed for 10 and 30 mg IMU-838.

Evaluation of safety parameters is shown in Table 6.

TABLE 6
Incidence of treatment-emergent adverse event (TEAE's)

| | | 10 mg | 30 mg |
|---|---|---|---|
| | Placebo | IMU-838 | IMU-838 |
| TEAEs | 44.4% | 35.6% | 40% |
| Serious TEAEs | 3.7% | 6.7% | 5% |
| TEAEs of Special Interest | 3.7% | 0.0% | 5% |
| Renal TEAEs | 7.4% | 2.2% | 5% |
| Liver TEAEs | 3.7% | 2.2% | 0.0 |

Set MP (N₁₀ = 45, N₃₀ = 40, N_{placebo} = 27); TEAE of special interest are defined as: red blood cells urine positive, hematuria or retroperitoneal pain

The incidence of TEAE's was lower in both 10 mg and 30 mg IMU-838 group than for placebo.

In summary, the use of IMU-838 during the maintenance phase was found to be safe and well tolerated. The incidence of TEAEs for treatment with IMU-838 was comparable to or slightly lower than placebo and very few serious TEAEs were observed. As compared to placebo, the incidence in the imu-838 treatment groups showed (detailed data not shown here): no increase in infections or infestations; no increased rate of liver events or liver enzyme elevations; no increased rate of renal events or AE of special interest; overall safety and tolerability profile of IMU-838 in UC patients is comparable to placebo.

### BRIEF DESCRIPTION OF THE DRAWINGS

FIG. 1: Flow chart of the CALDOSE-1 study according to Example 1, the induction phase is highlighted.

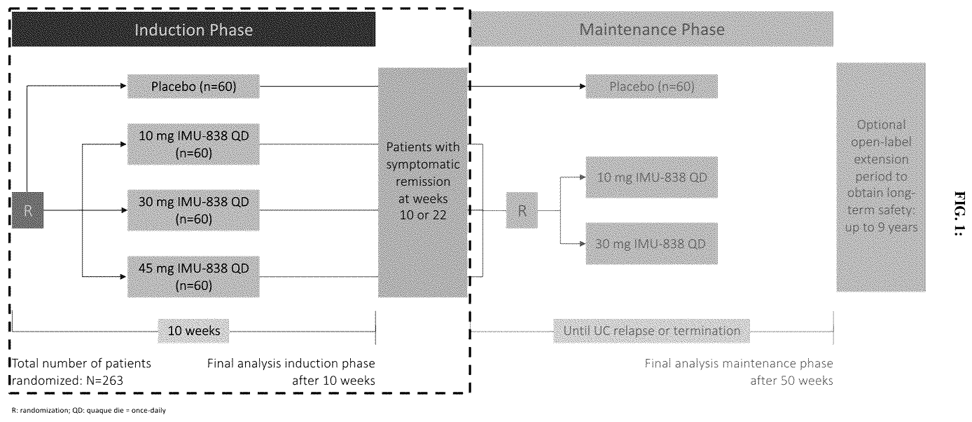

FIG. 2: Demographics i.e. stratification at randomization for prior biologics and concurrent corticosteroid use within the CALDOSE-1 study according to Example 1. The table provides the demographic information for prior biologics use and concurrent use of corticosteroids, as used as stratification factor for randomization as provided by the investigator. The actual rate of concurrent corticosteroid use may be slightly different than these strata information, however no substantial difference is expected. Allowed concurrent corticosteroids per protocol: Use of oral systemic corticosteroids (≤20 mg/day prednisolone equivalent), including beclomethasone dipropionate (at ≤5 mg/day) and budesonide (MMX at ≤9 mg/day) unless they have been used for at least four weeks before first randomization, and at a stable dose for at least two weeks before first randomization. Concurrent use of corticosteroids used as stratification factor at randomization (ensures equivalent distribution to all treatment groups). Doses of corticosteroids were required to be stable and could be changed throughout the induction phase.

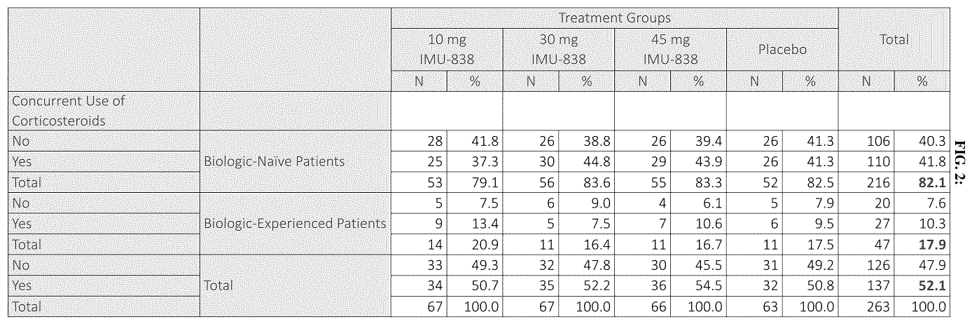

FIG. 3: Clinical remission values of the CALDOSE-1 study according to Example 1 indicating that the primary endpoint was not achieved (primary endpoint was based on Full Analysis Set: Pooled 30 and 45 mg IMU-838 data: 13.8%; 16/116 (n=16 missing data at Week 10), placebo: 14.0%; 8/49 (n=16 missing data at Week 10), not statistically significant; ITT: intent-to-treat).

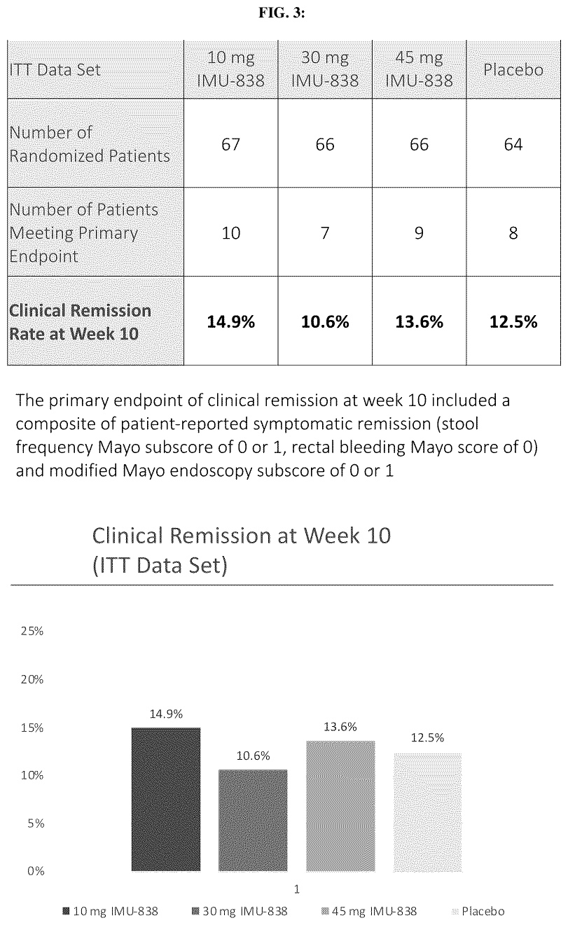

FIG. 4: Antagonistic effect between vidofludimus calcium (IMU-838) and concurrent use of corticosteroids (stratum from randomization) within the study according to Example 1 (the graphs use the concurrent use of corticosteroids, as used as stratification factor for randomization provided by the investigator, not actual use of concurrent corticosteroids. Data display ITT population of both biologic-naïve and -experienced patients. Pooled vidofludimus calcium data contain all data from 10 mg (no steroids n=33, steroids n=34), 30 mg (no steroids n=32, steroids n=34), 45 mg IMU-838 (no steroids n=30, steroids n=36). Placebo data: no steroids n=30, steroids n=34; Clinical Remission: achieving both symptomatic remission and endoscopic remission; Symptomatic Remission: Mayo rectal bleeding subscore of 0, and Mayo stool frequency subscore of 0 or 1; Endoscopic Remission: Modified Mayo endoscopy subscore of 0 or 1).

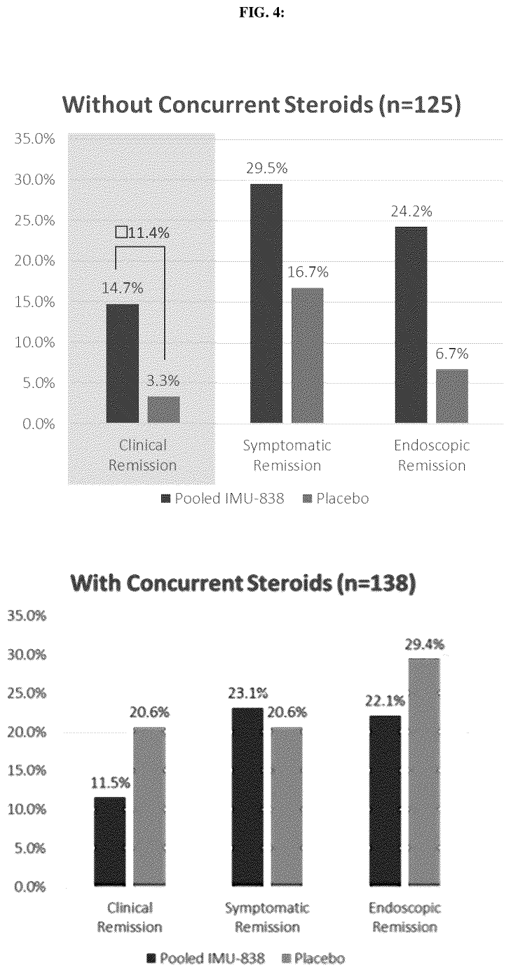

FIG. 5: Antagonistic effect between vidofludimus calcium (IMU-838) and concurrent use of corticosteroids (actual concurrent corticosteroid use stratum) within the study according to Example 1. (the graphs use the patient population regarding actual use of concurrent corticosteroids. data display ITT population as in FIG. 4).

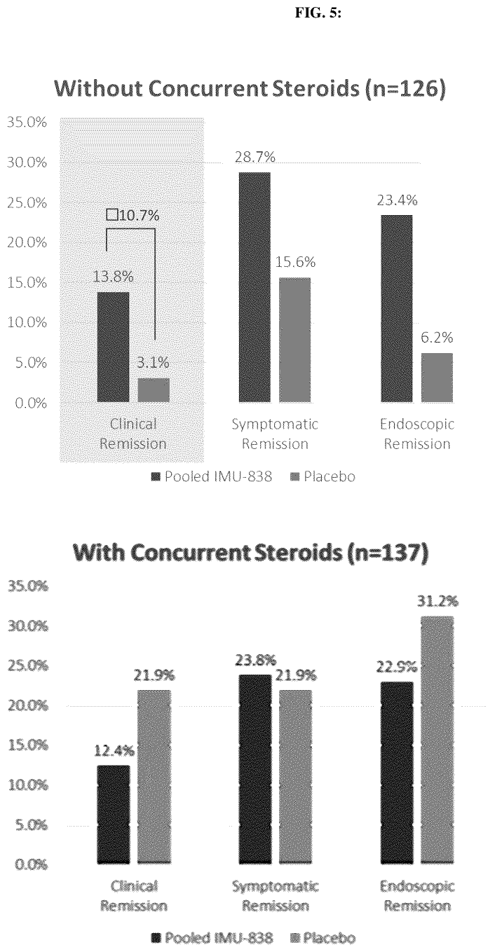

FIG. 6: Overview of beneficial effects in endoscopic healing, clinical remission and symptomatic response for different doses.

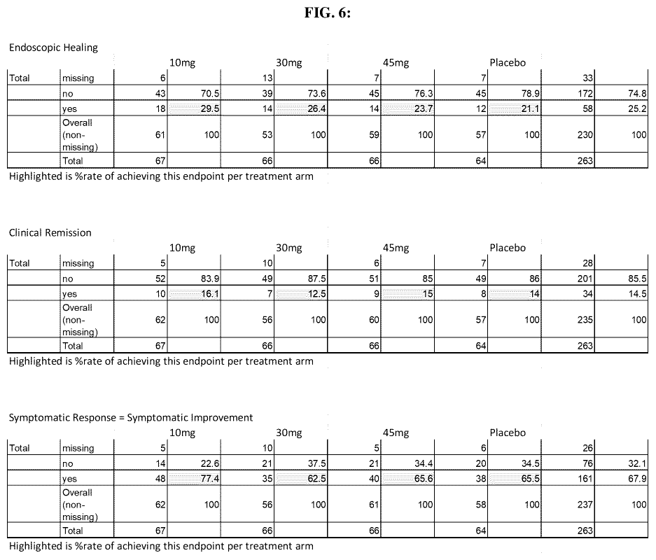

FIG. 7: Patient population in the maintenance phase (MP) for each treatment group.

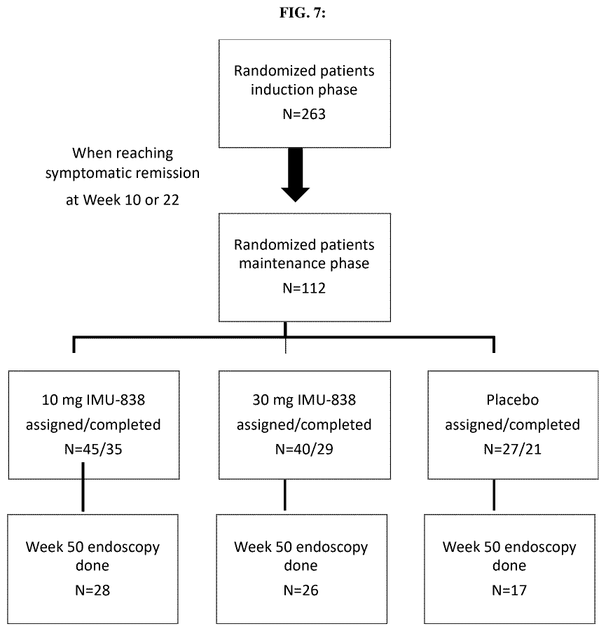

1-15. (canceled)

16. A method of treating ulcerative colitis in a human patient in need thereof, the method comprising orally administering to the human patient a daily dose of about 12 to about 127 μmol vidofludimus and/or a pharmaceutically acceptable salt and/or a solvate, in particular a hydrate, thereof and/or a solvate, in particular a hydrate, of a pharmaceutically acceptable salt thereof;
wherein the method further comprises administering to the human patient a systemic corticosteroid co-medication.

17. The method of claim 16, wherein the systemic corticosteroid co-medication is an oral systemic corticosteroid co-medication and is administered at 20 mg/day or less prednisolone equivalent.

18. The method of claim 17, wherein the oral systemic corticosteroid co-medication had been used for at least 4 weeks and with a stable dose for at least 2 weeks before administering vidofludimus.

19. The method of claim 16, wherein the human patient is afflicted with moderate to severe ulcerative colitis.

20. The method of claim 16, wherein treatment is for 52 weeks, whereby the human patient achieves clinical remission and wherein the human patient achieves an endoscopic response.

21. The method of claim 16, wherein the human patient is a responder after at least 10 weeks of treatment based on satisfying one or more clinical endpoints selected from the group consisting of:
(a) symptomatic remission;
(b) Mayo rectal bleeding subscore of 0;
(c) Mayo stool frequency subscore of 0 or 1; and
(d) Mayo endoscopy subscore of 0 or 1.

22. The method of claim 16, wherein the human patient is a responder after 52 weeks of treatment based on satisfying one or more clinical endpoints selected from the group consisting of:
(a) symptomatic remission;
(b) Mayo rectal bleeding subscore of 0;
(c) Mayo stool frequency subscore of 0 or 1; and
(d) Mayo endoscopy subscore of 0 or 1.

23. The method of claim 16, wherein vidofludimus is administered in the form of vidofludimus calcium salt dihydrate with the following structure: 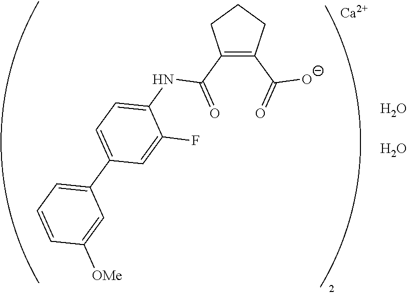
as Polymorph A in the form of a tablet or a capsule.

24. A method of treating ulcerative colitis in a human patient in need thereof, the method comprising orally administering to the human patient a daily dose of about 25.5 μmol vidofludimus and/or a pharmaceutically acceptable salt and/or a solvate, in particular a hydrate, thereof and/or a solvate, in particular a hydrate, of a pharmaceutically acceptable salt thereof.

25. The method of claim 24, wherein the human patient is afflicted with moderate to severe ulcerative colitis.

26. The method of claim 24, wherein treatment is for 10 weeks, whereby the human patient achieves clinical remission and wherein the human patient achieves an endoscopic response.

27. The method of claim 24, wherein the human patient is a responder after 10 weeks of treatment based on satisfying one or more clinical endpoints selected from the group consisting of:
(a) symptomatic remission;
(b) Mayo rectal bleeding subscore of 0;
(c) Mayo stool frequency subscore of 0 or 1; and
(d) Mayo endoscopy subscore of 0 or 1.

28. The method of claim 24, wherein the human patient achieves a higher symptomatic remission rate compared to a daily dose of 76.5 or 115 μmol of vidofludimus.

29. The method of claim 24, wherein vidofludimus is administered in the form of vidofludimus calcium salt dihydrate with the following structure: 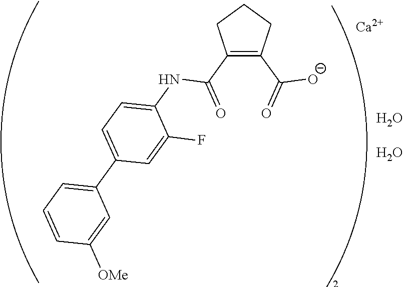
as Polymorph A in the form of a tablet or a capsule.

30. A method of treating ulcerative colitis in a human patient in need thereof, the method comprising orally administering to the human patient a daily dose of about 12 to about 127 μmol vidofludimus and/or a pharmaceutically acceptable salt and/or a solvate, in particular a hydrate, thereof and/or a solvate, in particular a hydrate, of a pharmaceutically acceptable salt thereof;
wherein the human patient is without or tapered systemic corticosteroid medication during administering vidofludimus.

31. The method of claim 30, wherein the human patient discontinued intravenous corticosteroid medication at least 4 weeks before administering vidofludimus.

32. The method of claim 30, wherein the human patient discontinued oral corticosteroid medication at least 2 weeks before administering vidofludimus.

33. The method of claim 30, wherein the human patient is afflicted with moderate to severe ulcerative colitis.

34. The method of claim 30, wherein the treatment is for 52 weeks, whereby the human patient achieves clinical remission and wherein the human patient achieves an endoscopic response.

35. The method of claim 30, wherein the human patient is a responder after at least 10 weeks of treatment based on satisfying one or more clinical endpoints selected from the group consisting of:
(a) symptomatic remission;
(b) partial Mayo remission;
(c) Mayo rectal bleeding subscore of 0;
(d) Mayo stool frequency subscore of 0 or 1;
(e) mean absolute stool numbers decreased by at least 3;
(f) decrease in corticosteroid usage and/or dosage;
(g) corticosteroid-free symptomatic remission;
(h) corticosteroid-free partial mayo remission;
(i) normalized fecal lactoferrin;
(j) normalization of fecal calprotectin levels;
(k) >16-point improvement from induction baseline in the total Inflammatory Bowel Disease Questionnaire (IBDQ) score; and
(l) IBDQ remission.

36. The method of claim 30, wherein the human patient is a responder after 52 weeks of treatment based on satisfying one or more clinical endpoints selected from the group consisting of:
(a) symptomatic remission;
(b) partial Mayo remission;
(c) Mayo rectal bleeding subscore of 0;
(d) Mayo stool frequency subscore of 0 or 1;
(e) mean absolute stool numbers decreased by at least 3;
(f) decrease in corticosteroid usage and/or dosage;
(g) corticosteroid-free symptomatic remission;
(h) corticosteroid-free partial mayo remission;
(i) normalized fecal lactoferrin;
(j) normalization of fecal calprotectin levels;
(k) >16-point improvement from induction baseline in the total Inflammatory Bowel Disease Questionnaire (IBDQ) score; and
(l) IBDQ remission.

37. The method of claim 30, wherein vidofludimus is administered in the form of vidofludimus calcium salt dihydrate with the following structure: 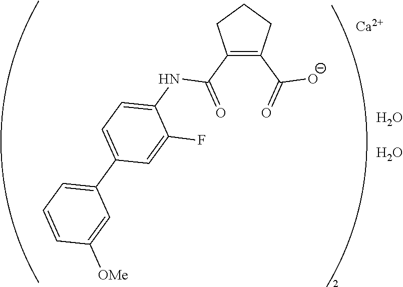
as Polymorph A in the form of a tablet or a capsule.
